(19) United States
(12) Patent Application Publication
(10) Pub. No.: US 2015/0258217 A1
(43) Pub. Date: Sep. 17, 2015

(54) Methods of Synthesizing and Using Peg-Like Fluorochromes

(71) Applicant: THE GENERAL HOSPITAL CORPORATION, Boston,, MA (US)

(72) Inventors: Peter Caravan, Charlestown, PA (US); Lee Josephson, Reading, MA (US); Yanyan Guo, Quincy, MA (US); Hushan Yuan, West Roxbury, MA (US)

(21) Appl. No.: 14/433,272

(22) PCT Filed: Sep. 19, 2013
(86) PCT No.: PCT/US2013/060565

§ 371 Date: Apr. 2, 2015

Related U.S. Application Data
(60) Provisional application No. 61/709,424, filed on Oct. 4, 2012.

(51) Int. Cl. A61K 49/00; A61K 51/04; A61K 47/48; A61K 51/08

(52) U.S. Cl. CPC A61K 49/0056; A61K 49/0054; A61K 49/0021; A61K 51/088; A61K 51/0482; A61K 47/48338; A61K 47/48215

(57) ABSTRACT

Fluorescent compounds include near infrared fluorochromes that are covalently linked to polyethylene glycol (PEG). The compounds behave like PEG in biological systems. One fluorescent compound has the formula (I): wherein R¹ is a fluorescent moiety having an absorption wavelength maxima in the range of 450 to 1500 nanometers, R² is a non-reactive moiety, and n is an integer. Another fluorescent compound has the formula (II): wherein R¹ is a fluorescent moiety having an absorption wavelength maxima in the range of 450 to 1500 nanometers, R² is a non-reactive moiety, R³ is a scaffold including an amino acid group, and n is an integer. The scaffold can be attached to a chelate, protein, enzyme, peptide, antibody, or drug that can target a site in a subject.

### CROSS-REFERENCES TO RELATED APPLICATIONS

This application claims priority from U.S. Patent Application No. 61/709,424 filed Oct. 4, 2012, which is incorporated by reference herein.

### STATEMENT REGARDING FEDERALLY SPONSORED RESEARCH

This invention was made with government support under grant number EB 009691 awarded by the National Institutes of Health. The government has certain rights in the invention.

### BACKGROUND OF THE INVENTION

1. Field of the Invention

The invention relates to uses and compositions of near infrared (NIR) fluorochromes that are covalently linked to polyethylene glycol (PEG), and behave like PEG in biological systems, including synthetic methods, compositions and methods using these PEG-like fluorochromes. The NIR fluorochromes are improved by becoming PEG-like, or behaving like PEG in biological systems, by which is meant they do not bind to cells, lipids or tissues unless through specific molecular interactions. In contrast, previously developed NIR fluorochromes, and materials made with them, interact strongly with cells, lipids and tissues.

2. Description of the Related Art

Fluorescent compounds play an essential role in molecular imaging both in vitro and in vivo. Of these fluorescent compounds, near infrared (NIR) fluorophores have ideal absorption/emission wavelengths between 550 and 1000 nanometers, which minimize autofluorescence interference from tissue and have minimal overlap with biological chromophores such as hemoglobin. Fluorophores in which NIR fluorochromes have been conjugated to peptides or nanoparticles have successfully been applied to in vivo imaging of tumors.

Existing NIR fluorochromes do have limitations. Though NIR fluorochromes are desirable for imaging in biological systems because of the tissue penetrating properties of their light, they are chemically complex structures involving multiple unsaturated double bonds linking multiple unsaturated rings. These features lead to self-quenching due to fluorochrome/fluorochrome interactions, high non-specific binding to many cells, unwanted interactions with proteins and lipids in vivo (high non-specific binding), and enterohepatic circulation rather than renal elimination. Fluorescence dye quenching can take place by dye stacking, which occurs when two or more fluorescence molecules are separated by a short-enough distance for their planar aromatic rings to interact to form aggregates. The absorbance spectra of dyes in a stacked state are substantially different from those of the same dye without stacking. For a description of the limitations of NIR fluorochromes, including the clinically used fluorochrome indocyanine green (ICG), see Choi et al., Synthesis and in vivo fate of zwitterionic near-infrared fluorophores. Angewandte Chemie 50, 6258-63 (2011).

Indocyanine green, a low molecular weight NIR fluorochrome that is currently widely used, binds to albumin, circulating lipoproteins and cell lipids, and is rapidly cleared to the liver by the hepatobiliary transport system of the liver. Though cleared with a blood half of 2-4 minutes, and indicated for determining hepatic function and angiography of the eye, intraoperative ICG angiography (aneurysm repair, flap patency) have nevertheless exploited ICG's non-ideal, short lived period of vascular contrast. For intraoperative fluorescent imaging two major limitations of ICG are: (i) a short blood half-life which limits vascular phase contrast to a few minutes post injection, and (ii) a high affinity for biomolecules that complicates efforts to use it as a probe of late phase (long time after injection), transcapillary passage/vascular permeability. This in turn limits the value of ICG in the key breast cancer application and is further discussed below.

Because the vast majority of ICG is tightly bound to biomolecules in vivo, ICG's transcapillary passage can occur as the free minority form of ICG, or as ICG bound to the various molecules to which it binds (e.g. 5 nm. albumin, 20 nm. lipoprotein). Efforts to analyze ICG's levels and disposition in tissues are also frustrated by its intense binding to biomolecules. Thus, when tissue fluorescence (i.e. interstitial fluorescence resulting from transcapillary passage) increases, both the mechanism of transcapillary transport and tissue levels of ICG cannot be ascertained. Others have recognized ICG's shortcomings and attempted to remedy them by synthesizing low molecular weight (M_{W}), ICG-like fluorochromes, one of which has been used clinically. These are not ideal because they retain many of ICG's limitations, particular protein binding, albeit to a lesser extent. ICG-like NIR fluorochromes have often been synthesized using a medicinal chemistry/organic chemistry approach and are reviewed in Table 1. For a further review of fluorochromes generally, see Luo et al., “A review of NIR dyes in cancer targeting and imaging”, Biomaterials 32, 7127-38 (2011).

TABLE 1
NIR (ICG-like) Fluorochromes Derived Using A Medicinal
Chemistry/Organic Chemistry Approach of Making Variations ICG

| Structure | References | Comment |
|---|---|---|
| Omociane 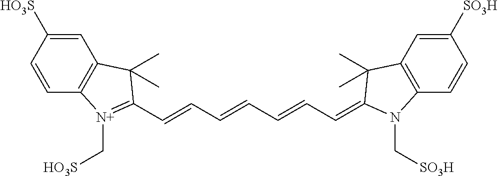 [SMILES: CC1(C)C(/C=C/C=C/C=C/C=C2/N(CS(=O)(=O)O)c3ccc(S(=O)(=O)O)cc3C2(C)C)=[N+](CS(=O)(=O)O)c2ccc(S(=O)(=O)O)cc21] | (See 1-4 below.) Also called SF-74 | Used clinically. |
| SIDAG 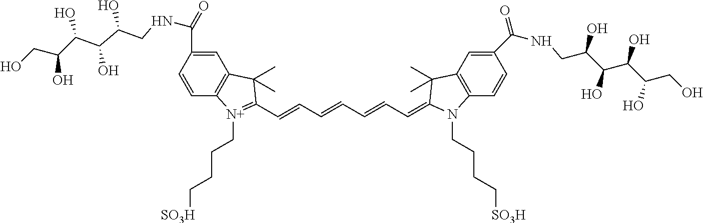 [SMILES: CC1(C)C(/C=C/C=C/C=C/C=C2/N(CCCCS(=O)(=O)O)c3ccc(C(=O)NC[C@@H](O)[C@H](O)[C@@H](O)[C@@H](O)CO)cc3C2(C)C)=[N+](CCCCS(=O)(=O)O)c2ccc(C(=O)NC[C@@H](O)[C@H](O)[C@@H](O)[C@@H](O)CO)cc21] | (See 1-3 below.) | |
| 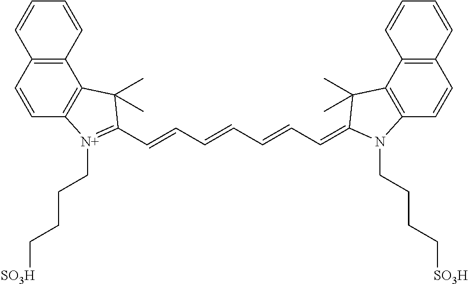 [SMILES: CC1(C)C(/C=C/C=C/C=C/C=C2/N(CCCCS(=O)(=O)O)c3ccc4ccccc4c3C2(C)C)=[N+](CCCCS(=O)(=O)O)c2ccc3ccccc3c21] ICG | (See 1-3 below.) | Widely used, very short blood half-life & high retention |
| 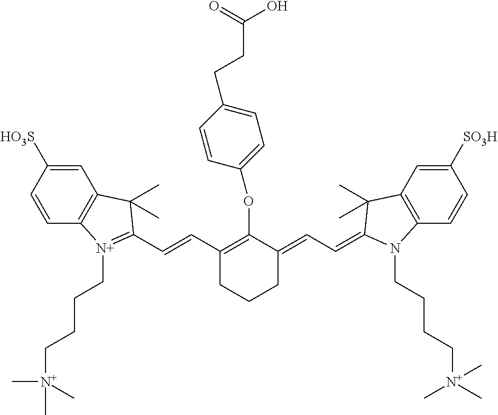 ZW800-1 | (See 5 below.) Research | |
| 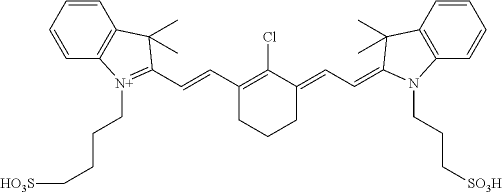 [SMILES: CC1(C)C(/C=C/C2=C(Cl)C(=C/C=C3/N(CCCS(=O)(=O)O)c4ccccc4C3(C)C)/CCC2)=[N+](CCCCS(=O)(=O)O)c2ccccc21] IR-783/Sigma | Research use only | |

References for Table 1:
(1) Ebert et al., (2011) Cyanine dyes as contrast agents for near-infrared imaging in vivo: acute tolerance, pharmacokinetics, and fluorescence imaging. Journal of biomedical optics 16, 066003.
(2) Perlitz et al., (2005) Comparison of two tricarbocyanine-based dyes for fluorescence optical imaging. Journal of fluorescence 15, 443-54.
(3) Licha et al., (2000) Hydrophilic cyanine dyes as contrast agents for near-infrared tumor imaging: synthesis, photophysical properties and spectroscopic in vivo characterization. Photochemistry and photobiology 72, 392-8.
(4) van de Ven, et al., (2010) A novel fluorescent imaging agent for diffuse optical tomography of the breast: first clinical experience in patients. Molecular imaging and biology: MIB: the official publication of the Academy of Molecular Imaging 12, 343-8.
(5) Choi et al., (2011) Synthesis and in vivo fate of zwitterionic near-infrared fluorophores. Angewandte Chemie (International ed. in English) 50, 6258-63.

There have been uses of PEG linkers between targeting molecules and fluorochromes. Bifunctional PEG's have been used as a linkers or spacers between fluorochromes and targeting biomolecules. One end of the PEG is reacted with a fluorochrome and the other with the targeting biomolecule. These designs employ the PEG to achieve a distance between the fluorochrome and targeting biomolecule and preserve the activity of the biomolecule, to increase size, to increase water solubility, and to facilitate purification. See Basilion, “An Optical Probe for Non-invasive Molecular Imaging of Orthotopic Brain Tumors Overexpressing Epidermal Growth Factor Receptor”, Molecular cancer therapeutics (2012); and Villaraza, “Improved speciation characteristics of PEGylated indocyanine green-labeled Panitumumab: revisiting the solution and spectroscopic properties of a near-infrared emitting anti-HER1 antibody for optical imaging of cancer”, Bioconjugate chemistry 21, 2305-12 (2010).

There have been uses of fluorochromes and PEG for enzyme activatable probes. Fluorochromes and PEG's have been used in the design of enzyme activated fluorescence probes. Such probes feature multiple PEG's and multiple fluorochromes per mole of probe to generate strong fluorochrome-fluorochrome interactions. The PEG's are bifunctional, having two reactive ends. Interactions between multiple fluorochromes on the probe produce quenching, which is alleviated when an enzyme hydrolyzes the probe. This generates fragment(s) with smaller numbers of fluorochromes per mole and a higher fluorescence.

However, the vast potential of intraoperative fluorescent imaging can only be realized when near infrared fluorochromes are developed which behave as discrete small molecules, that is, they do not bind albumin, cell membranes or lipid. Therefore, there is a need for hydrophilic (water loving, non-biomolecule binding, near infrared fluorescent) fluorochromes that can be clinically translated (simple to synthesize/low cost/pharmaceutically acceptable reactions).

### SUMMARY OF THE INVENTION

We have invented a new class of materials termed PEG-like NIR fluorochromes, and new methods of using PEG-like NIR fluorochromes, for diagnosis and treatment. The new materials can include a single PEG and a single fluorochrome that are covalently joined so that the fluorochrome is fully fluorescent (not quenched), and behaves like the PEG polymer in biological systems. PEG-like NIR fluorochromes can used as untargeted, intravenous injected intraoperative diagnostic agents. PEG-like fluorochromes can also be used as targeted, locally administered therapeutic agents.

In one aspect, the invention provides a fluorescent compound having the formula (I):

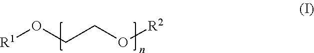

wherein R¹ is a fluorescent moiety having an absorption wavelength maxima in the range of 450 to 1500 nanometers, R² is a non-reactive moiety, and n is an integer.

In another aspect, the invention provides a fluorescent compound having the formula (II):

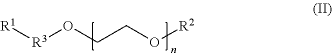

wherein R¹ is a fluorescent moiety having an absorption wavelength maxima in the range of 450 to 1500 nanometers, R² is a non-reactive moiety, R³ is a scaffold including an amino acid group, and n is an integer.

In yet another aspect, the invention provides a fluorescent compound having the formula (III):

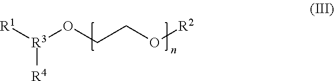

wherein R¹ is a fluorescent moiety having an absorption wavelength maxima in the range of 450 to 1500 nanometers, R² is a non-reactive moiety, R³ is a scaffold including an amino acid group, R⁴ is selected from chelates, proteins, enzymes, peptides, antibodies, and drugs that can target a site in a subject, and n is an integer.

In any of compounds (I), (II) or (III), n can be selected such that chain (C) in the compound

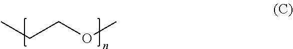

has a molecular weight of 2,000 daltons or more, or a molecular weight of 2,000 to 10,000 daltons, or a molecular weight of 5,000 to 40,000 daltons, or a molecular weight of 2,000 to 50,000 daltons, or a molecular weight of 2,000 to 100,000 daltons. Preferably, chain (C) in the compound shields R¹ (i.e., the fluorescent moiety) from reaction with biological molecules. In any of compounds (I), (II) or (III), n can be selected such that after intravenous administration of the compound (I), (II) or (III) to a mammal, the compound undergoes renal elimination. In any of compounds (I), (II) or (III), n can be selected such that after intravenous administration of the compound (I), (II) or (III) to a mammal, clearance is by macrophages of the reticuloendothelial system of the mammal.

The fluorescent moiety in any of compounds (I), (II) or (III) may have an absorption wavelength maxima in the range of 550 to 850 nanometers or in the range of 650 to 850 nanometers. The fluorescent moiety can be a cyanine dye. The fluorescent moiety can be a carbocyanine dye. The fluorescent moiety can be fluorescein. Any of the compounds (I), (II) or (III) can have a quantum yield of greater than 0.1.

Any of the compounds (I), (II) or (III) can have a molecular volume that correlates with an apparent molecular weight greater than about 10,000 daltons when analyzed by fast protein liquid chromatography and globular protein standards. Any of the compounds (I), (II) or (III) can have a molecular volume that correlates with an apparent molecular weight greater than about 20,000 daltons when analyzed by fast protein liquid chromatography and globular protein standards. Any of the compounds (I), (II) or (III) can have a molecular volume that correlates with an apparent molecular weight greater than about 30,000 daltons when analyzed by fast protein liquid chromatography and globular protein standards. Any of the compounds (I), (II) or (III) can have a molecular volume that correlates with an apparent molecular weight of about 10,000 daltons to about 30,000 daltons when analyzed by fast protein liquid chromatography and globular protein standards.

In any of compounds (I), (II) or (III), R² (i.e., the non-reactive moiety) can be selected from the group consisting of C₁-C₂₀ alkyl and aryl (e.g., phenyl). In any of compounds (I), (II) or (III), R² can be selected from the group consisting of C₁-C₅ alkyl.

In compound (III), R⁴ can be a chelate including a chelating agent and a chelated metal or metal ion. Example chelating agents are diethylene triamine pentaacetic acid (DTPA) or tetraazacyclododecane tetraacidic acid (DOTA) or desferoxamine (DFO). Preferably, the chelating agent is bifunctional, meaning that it possesses a metal binding moiety function and also possesses a separate chemically reactive functional group capable of covalently attaching to another moiety, such as a peptide. Non-limiting examples of bifunctional chelating agents that could be used include bifunctional DTPA, bifunctional DOTA, bifunctional DFO, bifunctional triazacyclononanetriacetic acid (NOTA), bifunctional tetraazabicyclopentadecatrienetriacetic acid (PCTA), and bifunctional oxatriazacyclododecanetriacetic acid (Oxo-DO3A). The chelated metal or metal ion in the chelate can be selected from Mn ions, Fe ions, gadolinium ions, ⁶⁷Ga, ⁶⁸Ga, ⁸²Rb, ⁸⁹Zr, ⁹⁰Y, ^{99m}Tc, ¹¹¹In, ¹⁷⁷Lu, ²⁰¹Tl, ²¹³Bi, and ²²⁵Ac. In some embodiments, a non-metal halogen, such as ⁷⁵Br, ⁷⁶Br, ¹⁸F, ¹²³I, ¹²⁵I, or ¹³¹I, may be bound to the chelated metal or metal ion. The chelate can include a magnetic material, or a paramagnetic material, or a superparamagnetic material. In one non-limiting example, the chelating agent is desferoxamine (DFO) and the metal is ⁸⁹Zr.

In any of compounds (I), (II) or (III), the compound can have a hydrodynamic diameter in the range of 1 to 100 nanometers or in the range of 2 to 50 nanometers or in the range of 1 to 20 nanometers or in the range of 3 to 15 nanometers or in the range of 4 to 11 nanometers.

In any of the compounds (II) or (III), the scaffold can be a peptide including two or more residues selected from alanine, arginine, aspartate, cysteine, glycine, and lysine. The peptide scaffold can include any number of residues; however, for ease of synthesis and reproducibility in clinical trials, it is preferred to limit the residues in the peptide to 20 or less, more preferably, 10 or less, more preferred 5 or less, and most preferred 3 or less. The scaffold can be attached to pharmacologically active groups, immunoreactive haptens, polymers, nanoparticles, proteins, enzymes, drugs, and vitamins. In one example form, the scaffold is attached to a protein, enzyme, peptide, antibody, or drug that can target a specific site (e.g., tumor) in a subject (human or animal) undergoing a diagnostic medical procedure.

In still another aspect, the invention provides a method for imaging a region of interest of a subject. The method comprises administering to the subject any of the compounds (I), (II) or (III), wherein the compound enters the region of interest of the subject; directing light into the subject; detecting fluorescent light emitted from the subject; and processing the detected light to provide an image that corresponds to the region of interest of the subject. The light directed into the subject can have a wavelength in the range of 450 to 1500 nanometers. Use of a fluorescent moiety having an absorption wavelength maxima in the range of 450 to 1500 nanometers and light having a wavelength in the range of 450 to 1500 nanometers maximizes tissue penetration and minimizes absorption by physiologically abundant absorbers such as hemoglobin and water. The fluorescent light may be emitted via two-photon-excited fluorescence. The method can further include imaging the subject with a second imaging method selected from positron emission tomography, single-photon emission computed tomography, magnetic resonance imaging, computerized tomography, optical imaging, and ultrasound. The region of interest of the subject may include a tumor. If the compound binds to the tumor, the method can further comprise administering to the subject a therapeutically effective amount of a cytotoxic material comprising any of the compounds (I), (II) or (III) associated with a cytotoxic agent.

In yet another aspect, the invention provides a method for treatment of a tumor in a subject. The method comprises administering to the subject a therapeutically effective amount of a cytotoxic material comprising any of the compounds (I), (II) or (III) associated with a cytotoxic agent. The cytotoxic material is targeted to the tumor in the subject.

In still another aspect, the invention provides a method for treatment of a tumor in a subject. The method comprises administering to the subject a therapeutically effective amount of a cytotoxic material comprising any of the compounds (I), (II) or (III) associated with a cytotoxic agent, wherein the cytotoxic material is targeted to the tumor in the subject. Preferably, the cytotoxic material is injected peritumorally, and at least a portion of the cytotoxic material is retained at or near the tumor by interactions between a scaffold of the compound and a receptor on a surface of a cell in the tumor.

In one version, the invention provides a composition of matter consisting (exclusively) of a NIR fluorochrome and a PEG.

In another version, the invention provides a composition of matter consisting of a single amino acid, a NIR fluorochrome and a PEG.

In yet another version, the invention provides a composition of matter consisting of a chelator, a PEG, and fluorochrome attached to a single amino acid.

In still another version, the invention provides a method of diagnostic imaging employing a passively targeted probe, PEG-like fluorochrome compound which is intravenously injected, and an image of the fluorescence in an animal or human is obtained, where the PEG-like fluorochrome consists of (i) a single NIR fluorochrome per mole and (ii) a single PEG per mole, the PEG being larger than about 2 kDa, and which blocks fluorochrome-fluorochrome mediated interactions or fluorochrome-biomolecule interactions.

In yet another version, the invention provides a method of tumor therapy employing a probe consisting of a PEG-like fluorochrome, where the PEG-like fluorochrome consists of (i) a single NIR fluorochrome per mole and (ii) a single PEG per mole, the PEG being larger than about 2 kDa, and a targeting vehicle that is locally injected, allowed to diffuse through the interstitium, and retained at or near the tumor by interactions between the targeting vehicle component of the probe and a receptor on the surface of cell in a tumor.

These and other features, aspects, and advantages of the present invention will become better understood upon consideration of the following detailed description, drawings, and appended claims.

### BRIEF DESCRIPTION OF THE DRAWINGS

FIG. 1 shows a strategy that was used to synthesize peptides having a polyethylene glycol chain and a fluorochrome according to certain example embodiments of a fluorescent compound of the invention.

FIG. 2 shows the synthesis of a linker peptide targeting vehicle suitable for use in certain example embodiments of a fluorescent compound of the invention.

FIG. 3 shows the synthesis of trifunctional probes according to certain example embodiments of a fluorescent compound of the invention.

FIG. 4A shows a comparative example (5a) with respect to a fluorescent compound of the invention.

FIG. 4B shows an example embodiment (5b) of a fluorescent compound of the invention.

FIG. 4C shows a comparative example (6a) with respect to a fluorescent compound of the invention.

FIG. 4D shows a comparative example (6b) with respect to a fluorescent compound of the invention.

FIG. 4E shows an example embodiment (7a) of a fluorescent compound of the invention.

FIG. 4F shows an example embodiment (7b) of a fluorescent compound of the invention.

FIG. 5 shows how a peptide scaffold, bearing PEG and a fluorochrome is attached to a targeting group which binds to a molecular target expressed by a cell within a tumor.

FIG. 6 shows an example embodiment of a radioisotope labeled fluorescent compound (9a, b) of the invention.

FIG. 7 depicts a comparison of intravenous administration and diffusion molecular retention according to an aspect of the invention.

FIG. 8 shows tumor targeting in two animals by DMR by using the GFP expressing BT-20 breast carcinoma xenograft by surface fluorescence.

FIG. 9 shows the efficiency of tumor targeting by DMR or IV methods.

FIG. 10 shows SPECT/CT images after DMR and IV injections with an ¹¹¹In RGD probe using a BT-20 tumor model, and shows tissue radioactivity concentrations obtained with the RAD and RGD probes.

FIG. 11 shows a general synthesis of PEG-like fluorochromes on a dipeptide scaffold.

FIG. 12 shows general methods (a, b, c) of reacting a PEG and a fluorochrome with an amino acid.

FIG. 13 shows two general strategies for reacting fluorochromes and polyethylene glycol according to an aspect of the invention.

FIG. 14 shows three general methods of directly reacting a PEG with a fluorochrome.

FIG. 15 shows a scheme for the synthesis of a (DOTA)-Lys-Cys peptide.

FIG. 16 shows a scheme for the synthesis of a (DOTA)Lys-Cys(IR-783) peptide.

FIG. 17 shows a scheme for the synthesis of a (DOTA)Lys-Cys(Cy3) peptide.

FIG. 18 shows a scheme for the synthesis of a (DOTA)Lys-Cys(Fluorescein) peptide.

FIG. 19 shows a scheme for the synthesis of a (DOTA)Lys(PEG)-Cys(IR-783) peptide.

FIG. 20 shows a scheme for the synthesis of a (DOTA)Lys(PEG)-Cys(Cy3) peptide.

FIG. 21 shows a scheme for the synthesis of (DOTA)Lys(PEG)-Cys(Fluorescein) peptide.

FIG. 22 shows the synthesis and design principles of PEG-like Nanoprobes (PN's). In FIG. 22(a), a modular synthetic strategy is employed with one fixed component, the (DOTA)Lys-Cys peptide. A variable fluorochrome reacts with the cysteine side chain, followed reaction of an NHS ester of a PEG polymer, of variable length, with the lysine side chain. FIG. 22(b) shows conferring “PEG-likeness” with a long PEG polymer and short peptide. PEG confers its size upon the resulting probe. Spectral tunability is generated by fluorochrome selection. Pharmacokinetic tunability is generated by PEG selection. In FIG. 22(c), PEG-likeness enhances fluorochrome elimination: Surface fluorescence of mice after IV injections of the (DOTA)Lys-Cys(IR-783) peptide or PN(783)4.3, which is a PEGylated version of the same peptide. PEGylation leads to enhanced fluorochrome elimination, which is evident by the selective fluorescent bladder at 20 minutes.

FIG. 23 shows tuning PN size, optical properties and the post-injection circulating form. In FIG. 23(a), tuning PN size by varying PEG. FPLC chromatograms of size-variable PN's (constant IR-783 fluorochrome, variable PEG's, see FIG. 22a) are shown. Volumes are given in Table 5. FIG. 23(b,c) shows tuning size with different fluorochromes. The 5 kDa PEG yielded the magenta coded PN chromatograms with diameters of 4.3 nm regardless of the fluorochrome used. They are PN(783)4.3 (2a), PN(545)4.3 (2b), and PN(497)4.3 (2c). The 30 kDa PEG yielded the blue coded PN's of 10 nanometers. PN dimensions are determined by PEG and independent of the fluorochrome selected. In FIG. 23(d,e,f), FPLC chromatograms of PN's are shown before (pre) and at various times after injection. PN's were PN(783)10 (FIG. 23d), PN(783) 6.1 (FIG. 23e), and PN(783)10.0 (FIG. 23f). After injection, PN's circulate at their PEG-determined and variable pre-injection sizes. In FIG. 23(g), since PN's circulate at PEG-determined, pre-injection sizes, they cross capillaries at those sizes.

FIG. 24 shows tuning PN Pharmacokinetics analyzed by the two-compartment pharmacokinetic model. FIG. 24(a) shows a summary of the two compartment pharmacokinetic model showing three microscopic rate constants. Serum fluorescence for PN(783)10 (b) and PN(783)4.3 (c) after injection are shown. Data were fit to the two compartment model shown in FIG. 24(a). In FIG. 24(d), post injection time courses of blood fluorescence for PN(783)10 (g) and PN(783)4.3 (h) are shown. Lines are the fits to a two compartment pharmacokinetic model with constants provided in Table 5.

FIG. 25 shows fluorescent imaging of three pharmacokinetic phases with PN's with diameters of 10 nm. FIG. 25(a) shows vascular phase, two-photon microscopy of brain vasculature. Vessel intensity drops due vascular escape, but there is no interstitial fluorescence in the brain due to the blood brain barrier. Scale marker=50 microns. FIG. 25 (b) shows intravital confocal microscopy intravital of the vascular and interstitial phases of an mCherry expressing HT-29 xenograft. During the vascular phase (10 minutes post) injection, vessels are imaged, without interstitial fluorescence. During the interstitial phase (20 hours post), interstitial fluorescence is prominent. Scale marker=20 microns. FIG. 25 (c) shows confocal microscopy of the tumor retention phase of PN(497)10.0. Shown are a sectioned HT-29 mCherry expressing tumor with nuclei stained blue (DAPI), mCherry tumor cells (red), PN(497)10.0 (green) and a green/red overlay (yellow). FIG. 25 (d) shows surface fluorescence/X-ray imaging of the tumor retention phase of PN(545)10.0. Shown are the HT-29/mCherry tumor with the skin removed as a white light image, mCherry tumor fluorescence (green), PN(545)10.0 fluorescence (purple) and the green/purple over lay (white).

FIG. 26 shows multimodal imaging of tumor retention with PN(783)10.0, its biodistribution and elimination. FIG. 26(a) shows SPECT/CT images of two mice bearing two HT-29 tumors as a function of time after injection. At two hours post injection, agent is in the blood and interstitium. By 24 hours post injection, tumors are becoming apparent as agent is being cleared. At 48 hours, labeling is highly tumor selective. FIG. 26(b) shows surface fluorescence imaging of two additional mice bearing the same tumor. By surface fluorescence, as with SPECT, labeling is highly tumor selective at 48 hours. Biodistribution as organ radioactivity concentrations FIG. 26(c) and total organ radioactivity FIG. 26(d) were obtained by dissection and ¹¹¹In counting at 24 hours and 48 hours post injection. Even at 48 hours post injection some 7.5% of injected dose is in the blood, with less than 5% in liver, even though the diameter of PN(783)10.0 exceeds that of albumin (6.7 nm). Data are means and standard deviations. FIG. 26(e) shows a whole animal radioactivity elimination cure. By 72 hours, some 17% of injected dose was retained, approximately half of which was still in the blood based on FIG. 26d.

FIG. 27 shows the purity of PEG-like Nanoprobes (PN's) by FPLC and mass spectroscopy. FIG. 27a) shows FPLC chromatogram of purification of PN(783)10.0 by removal of the low molecular weight (DOTA)Lys-Cys(IR-783) peptide which is used in PEG-like nanoprobe synthesis. FPLC's of pure PN's are shown in FIG. 23. FIG. 27b) shows MALDI-TOF Mass spectroscopy of pure PN(783)4.3 made by reaction of (DOTA)Lys-Cys(IR-783) with the 5 kDa PEG-NHS. Note the absence of species at 4800 to 5200 Da, expected if there was PEG contamination.

FIG. 28 shows a scheme for the synthesis of (¹¹¹In-DOTA)Lys(PEG30 kDa)-Cys(IR-783).

FIG. 29 shows quantum yields of peptides and PEGylated PN's. In FIG. 29a), quantum yields are shown for PN(783)'s made with different PEG's. In FIG. 29b), quantum yields are shown for PN(545)'s made with different PEG's. In FIG. 29c), quantum yields are shown for PN(497) made with different PEG's. As excitation maxima go up quantum yields go down. Quantum yields are always improved by PEGylation but the degree of improvement varies.

FIG. 30 shows the effect of PEGylation on non-specific binding to cells. In FIG. 30a), the (DOTA)Lys-Cys(IR-783) peptide binds cells but PEGylated versions have greatly reduced binding. The percent of cells with fluorescence higher than unstained cell is given in Table 5. In FIG. 30b), the (DOTA)Lys-Cys(Cy3) peptide binds to cells but PEGylated versions have greatly reduced binding. In FIG. 30c), the (DOTA)Lys-Cys(Fluorescein) peptide binds cells very weakly so PEG does not reduce binding. Arrows indicates the border of negative and positive binding.

FIG. 31 shows a scheme for the synthesis of a (DFO)Lys-Cys peptide wherein DFO is desferoxamine.

FIG. 32 shows a scheme for the synthesis of a (DFO)Lys-Cys(S-Mal-Cy5.5, Lumiprobe) peptide.

FIG. 33 shows a scheme for the attachment of a 5 kDa PEG to the peptide of FIG. 32.

FIG. 34 shows a scheme for the attachment of a 30 kDa PEG to the peptide of FIG. 32.

FIG. 35 shows a scheme for the radiolabeling of (DFO)Lys(PEG 30 kDa)-Cys(S-Mal-Cy5.5) with ⁸⁹Zr⁴⁺.

FIG. 36 shows radioactive thin-layer chromatography monitoring for DFO-Lys(PEG5KDa)-Cys(S-Mal-Cy5.5)-NH2.

FIG. 37 shows radioactive thin-layer chromatography monitoring for DFO-Lys(PEG30KDa)-Cys(S-Mal-Cy5.5)-NH2.

### DETAILED DESCRIPTION OF THE INVENTION

We have discovered that the covalent linking of a PEG and NIR fluorochrome can lead to a loss of unwanted fluorochrome-fluorochrome interactions (which lead to quenching) and unwanted fluorochrome-biomolecule interactions which lead to non-specific binding to plasma proteins, lipoproteins, cell membranes. PEG covers the fluorochrome with an extended polymeric cloud, with entrapped water, shielding it from reaction with biological molecules. Hence PEG-like fluorochromes are PEG-fluorochrome shielded fluorochromes.

For diagnostic imaging, the intravenous administration of a passively targeted PEG-like fluorochrome is obtained. With low molecular weight PEG's (2-10 kDa by mass), the PEG-like fluorochrome undergoes renal elimination (small enough for glomerular filtration). With high molecular weight PEG's (>20 kDa), clearance is by macrophages of the reticuloendothelial system (too large for glomerular filtration). Passively targeted PEG-like fluorochromes are used as intraoperative diagnostic agents to determine blood vessel flow or permeability. Images are made with fluorescent detection devices (cameras) such as those listed in Table 1 of Marshall, “Near-Infrared Fluorescence Imaging in Humans with Indocyanine Green: A Review and Update”, The Open Surgical Oncology Journal 2, 12-15 (2010). See also Alander, “A review of indocyanine green fluorescent imaging in surgery”, International journal of biomedical imaging 2012, 940585 (2012).

Some advantageous features of PEG-like fluorochromes over the conventional low molecular weight fluorochromes, which are listed in Table 1 above, are summarized below.

1. Because PEG-like fluorochromes exist as discrete species in biological systems (they do not interact with each other or biological molecules), their properties can be optimized for different intraoperative applications. PEG-like fluorochromes can be small (10 kDa) or large (e.g. 100 kDa), depending on the size of the PEG employed. The size of PEG-like fluorochromes can be varied to optimize their behavior as (i) angiographic agents (agents confined to the vasculature), (ii) as agents for visualizing transcapillary passage/vascular leak, and (iii) as agents for visualizing macrophages of the reticuloendothelial system.

2. The PEG-like fluorochromes use clinically translatable chemistry. The three basic components of PEG-like fluorochromes (i.e., PEG, fluorochrome, and amino acid or peptide) are inexpensive. The synthesis of PEG-like fluorochromes on a large scale is practical and consistent with pharmaceutical practice.

3. The PEG-like fluorochromes allow for detection by a second imaging modality. Our design allows the addition of a metal chelating functional group (e.g., a chelating agent such as diethylene triamine pentaacetic acid (DTPA), tetraazacyclododecane tetraacidic acid (DOTA), or desferoxamine (DFO)), and a chelated metal or metal ion (such as Mn ions, Fe ions, gadolinium ions, ⁶⁷Ga, ⁶⁸Ga, ⁸²Rb, ⁸⁹Zr, ⁹⁰Y, ^{99m}Tc, ¹¹¹In, ¹⁷⁷Lu, ²⁰¹Tl, ²¹³Bi, and ²²⁵Ac) to the PEG-like fluorochrome. Together, the chelating agent and the chelated metal or metal ion form a chelate. The presence of the chelate enables the PEG-like fluorochrome to be quantified by magnetic resonance imaging (MRI), positron emission tomography (PET), or single-photon emission computed tomography (SPECT), in addition to fluorescence. The multimodal capability can be used clinically or to accelerate the development of PEG-like fluorochromes for intraoperative applications by providing a method of measuring fluorochrome levels in tissues. To facilitate the incorporation of the chelate into the PEG-like fluorochrome, the chelating agent is preferably bifunctional, meaning that it includes both a metal chelating function and a separate active functional group that can covalently bond to other groups, such as a peptide. Exemplary bifunctional chelating agents that could be used include without limitation bifunctional DTPA, bifunctional DOTA, bifunctional DFO, bifunctional NOTA, bifunctional PCTA, and bifunctional Oxo-DO3A. Further non-limiting examples of bifunctional chelating agents that could be used in the invention are provided by Brechbiel (see Brechbiel M W. Bifunctional chelates for metal nuclides. Q J Nucl Med Mol Imaging. 2008 June; 52(2):166-73), which is incorporated by reference herein. Optionally, the invention can employ additional elements that are not metals by indirect chelation. After the metal or metal ion is chelated to the chelating agent, a non-metal halogen, such as ⁷⁵Br, ⁷⁶Br, ¹⁸F, ¹⁹F, ¹²³I, ¹²⁵I, ¹³¹I, may be bound to the chelated metal or metal ion, adding additional detection functionality. For example, McBride et al. have reported the binding of the halogen 18F to chelated aluminum ions to form a fluoride-aluminum-chelate complex (see McBride W J, D'Souza C A, Sharkey R M, Karacay H, Rossi E A, Chang C H, Goldenberg D M. 18F labeling of peptides with a fluoride-aluminum-chelate complex. Bioconjug Chem. 2010 Jul. 21; 21(7):1331-40. doi: 10.1021/bc100137x).

4. The invention provides the ability to use different fluorochromes. Since PEGylation enshrouds the fluorochrome, the fluorochrome can be varied while maintaining the PEG-like properties. This can allow the simultaneous use of two, spectrally distinct PEG-like fluorochromes (e.g., small and large PEG-like fluorochromes).

5. The invention provides pharmacokinetic (PK) control by changing PEG molecular weight and size. Unlike the fluorochromes of Table 1, where PK is intrinsic to the fluorochrome, the PK of PEG-like fluorochromes can be altered through alterations in the molecular weight and size of PEG.

6. The invention provides pharmacokinetic (PK) control by employing PEG to block proteolytic degradation. PEGylation can also PK control by blocking the degradation of fluorochrome bearing peptide by proteases. The degradation of peptides often occurs when then leave the vasculature and encounter proteases. PEGylation can provide PK control by blocking proteolytic degradation.

For cancer treatment, PEG-like probes are used with a molecular targeted delivery method called Diffusion Molecular Retention (DMR). DMR probes can use a short PEG linker as well as a larger PEG for fluorochrome shielding. Here PEG-like fluorochromes can be components of more complex probes that include molecular targeting groups and cytotoxic agents. Thus, a cytotoxic agent can be associated with a PEG-like fluorochrome.

A cytotoxic agent is “associated” with one of the PEG-like fluorochromes of the invention if the cytotoxic agent is directly or indirectly, physically or chemically bound to one of the PEG-like fluorochromes. Non-limiting examples of chemical bonds include covalent bonds, ionic bonds, coordinate bonds, and hydrogen bonds. Indirect bonding can include the use of a group of atoms (i.e., a linker) that chemically links the cytotoxic agent and the PEG-like fluorochrome. Non-limiting examples of physical bonding include physical adsorption and absorption. The cytotoxic agent can be a cytotoxin (e.g., ricin, pseudomonas exotoxin, diphtheria toxin). The cytotoxic agent can be a chemotherapeutic agent (e.g., alkylating agents, antagonists, plant alkaloids, intercalating antibiotics, enzyme inhibitors, antimetabolites, mitotic inhibitors, growth factor inhibitors, cell cycle inhibitors, enzymes, biological response modifiers). The cytotoxic agent can be a radiation-emitter (e.g., phosphorus-32, phosphorus-33, bromine-77, yttrium-88, yttrium-90, molybdenum-99m, technetium-99m, indium-111, indium-131, iodine-123, iodine-124, iodine-125, iodine-131, lutetium-177, rhenium-186, rhenium-188, bismuth-212, bismuth-213, astatine-211).

The molecularly targeted delivery of toxic “payloads” to tumors is limited by low tumor blood flow, capillary permeability barriers, high interstitial pressure, and kidney, liver and spleen uptake. The technique termed Diffusion Molecular Retention (DMR) comprises local administration of a fluorescent peptide probe, visualizing probe extensive probe diffusion through the interstitium by fluorescence, and obtaining retention if the probe encounters a molecular target. In this instance, PEG-like fluorochromes function as reporters of the interstitial diffusion of locally administered, targeted therapeutic PEG-like fluorochromes. DMR employs peptide probes that by virtue of their PEGylation achieve a molecular volume of 25 kDa, and therefore have a slow vascular uptake, as well as an absence of non-specific binding to components of the interstitium. To demonstrate DMR, a trifunctional RGD probe bearing a DOTA, a 5 kDa PEG and a CyAl5.5 fluorochrome was synthesized and interstitial diffusion visualized by surface fluorescence. A control RAD probe was not retained, indicating retention of the RGD probe was due to integrin binding. By “local administration” is meant intratumorally, peritumorally or with subcutaneous or intramuscular injections that enable the probe to diffuse to and through the tumor with high efficiency (low uptake by normal organs like the liver, kidney and spleen).

Methods of using PEG-like fluorochromes as diagnostic imaging agents and for DMR cancer treatment are summarized in Table 2.

TABLE 2
Summary of Methods of Using PEG-like Fluorochromes for Diagnostic Imaging and DMR Tumor Therapy

| | | | Roles | Benefit/ |
|---|---|---|---|---|
| Use | Administration | Targeting | of PEG | Uses |
| Intraoperative | Intravenous (IV) | Passive, | 1. Shield fluorochrome, block | Intraoperative visualization of vessel |
| diagnostic | | Size | unwanted non-specific interactions | fluorescence function and vessel |
| imaging | | based | 2. Provide size-based | permeability. See Example 3-5 below. |
| | | | pharmacokinetic control of | |
| | | | fluorescence after injection. Renal | |
| | | | elimination (low M_{W}) or | |
| | | | reticuloendothelial system | |
| | | | elimination (high M_{W}) | |
| Therapeutic | Intratumor (IT) | Active, | 1. Shield fluorochrome, block | High efficiency tumor targeting (low normal |
| Targeting | Peritumor (PT) | (binding | unwanted non-specific interactions | organ uptake) of radioisotopes or other |
| (DMR) | Intramuscular (IM) | to | 2. Provide a size (volume) optimal | highly toxic “payloads.” See Examples 1 and |
| | Subcutaneous (SC) | molecular | for high interstitial diffusion | 2 below. |
| | | target) | 3. Retention only due to molecular | |
| | | | target binding, no non-specific | |
| | | | binding | |

Two raw materials for the synthesis of PEG-like fluorochromes are monofunctional PEG's, preferably with molecular weights of 2000 daltons or greater, and NIR fluorochromes.

The PEG's used by invention are monoreactive, with one end connected to the fluorochrome (directly or indirectly) and the other non-reactive end of the PEG unmodified. (Hence, the PEG's used for fluorochrome shielding do not serve as linkers.) PEG's must be sufficiently long to block the chemical properties of the fluorochrome. Generally, they must have molecular weights of about 2000 Da or greater and can be monodisperse (single molecular weight species) or polydisperse. Currently, PEG's of 2000 Da or greater are generally polydisperse. The NIR fluorochromes used have absorption wavelength maxima of 450 nanometers to 1500 nanometers, and must at least be site amenable to chemical modification.

Non-limiting examples of suitable NIR fluorochromes are Cy5.5, Cy5, CyAL-5, CyAL5.5, and IR-783. CyAL-5 and CyAL5.5 are carbocyanine dyes described in United States Patent Application Publication No. 2011/0286933, which is incorporated herein by reference. CyAL-5 and CyAL5.5 are available from Molecular Targeting Technologies, Inc., West Chester, Pa., USA. IR 783 is cyanine dye available from Sigma Aldrich, St. Louis, Mo., USA. It is 2-[2-[2-Chloro-3-[2-[1,3-dihydro-3,3-dimethyl-1-(4-sulfobutyl)-2H-indol-2-ylidene]-ethylidene]-1-cyclohexen-1-yl]-ethenyl]-3,3-dimethyl-1-(4-sulfobutyl)-3H-indolium hydroxide, inner salt sodium salt.

Preferably, PEG-like fluorochromes probes employ a single PEG per mole of probe to enshroud the fluorochrome and a single fluorochrome per mole. Thus, the probes of the invention do not employ intramolecular quenching and are not activated by enzymes. In some cases a second, short PEG can be employed as a linker between a targeting peptide and the PEG used to enshroud the NIR fluorochrome.

PEG-like fluorochromes of the invention (both actively or passively targeted) have one or more of the following chemical properties: (i) they have one fluorochrome per mole; (ii) they have one PEG per mole; (iii) the PEG has a molecular weight greater than about 2000 Da; (iv) the PEG is monofunctional (has only one chemically reactive end); (v) they have molecular volumes greater than about 10 kDa when analyzed by fast protein liquid chromatography (FPLC) and globular protein standards, and their volume is comprised mostly of the volume of the PEG rather than the fluorochrome, i.e., without PEG the fluorochrome has a volume of less than about 2 kilodaltons; (vi) they have characteristic, unstacked absorption spectra; and (vii) they have improved quantum yields (in PBS) over non-PEGylated fluorochrome.

Preferably, PEG-like fluorochromes of the invention have one or more of the properties below when interacting with biological systems: (i) they have low non-specific bindings with cultured cells; (ii) they can undergo clearance (by surface fluorescence) from a local intramuscular (IM) injection site within 24 hours; and (iii) when probe volumes are below about 30 kDa, they undergo predominant renal elimination after intravenous injection.

PEG and an NIR fluorochrome can be combined by direct attachment. Alternatively, PEG and the NIR fluorochrome can be attached to a low molecular weight scaffold (e.g., an amino acid or a peptide), yielding compositions comprising a fluorochrome, scaffold and PEG. Some amino acids (e.g., lysine, cysteine, aspartate) can accommodate a PEG, a chelate and a fluorochrome.

For targeted uses for PEG-like fluorochromes, an example embodiment employs a probe made according to the methods of U.S. Patent Application Publication No. 2011/0159566, which is incorporated herein by reference. To obtain a therapeutic method of treating a tumor, the multifunctional probe is locally administered (peritumorally or subcutaneously) and bears a cytotoxic agent such as a radiation emitter.

The invention is further illustrated in the following Examples which are presented for purposes of illustration and not of limitation.

### EXAMPLES

### Example 1

Fluorochromes were attached to peptides both with and without PEG. Table 3A below provides a summary of the Example 1 compounds.

### Tetrapeptide Probe Synthesis

### Overall Strategy

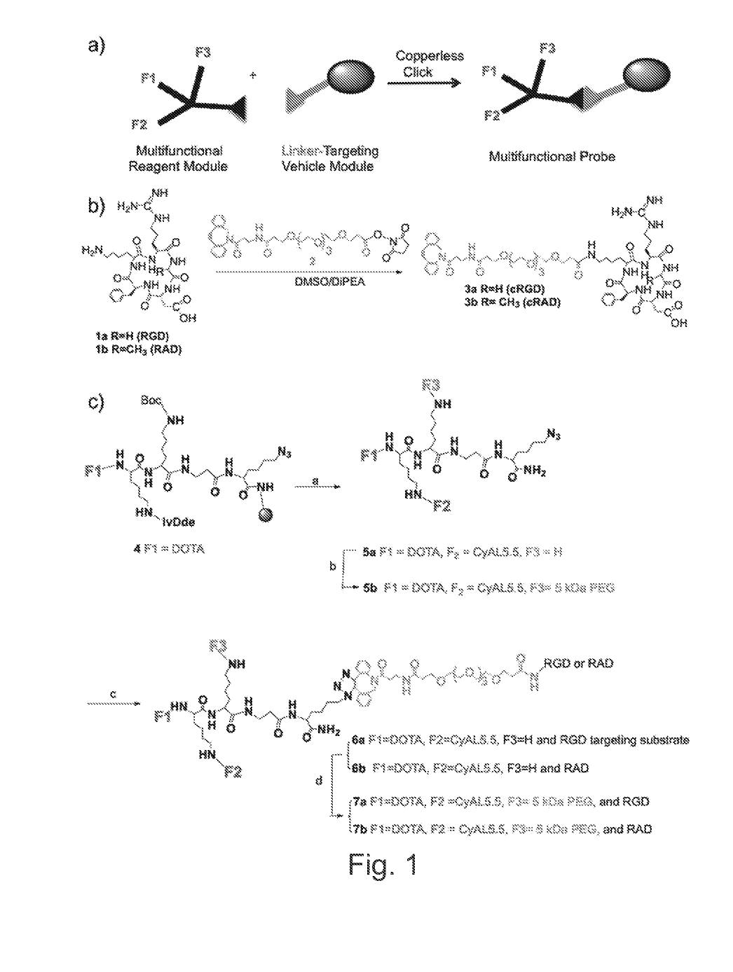

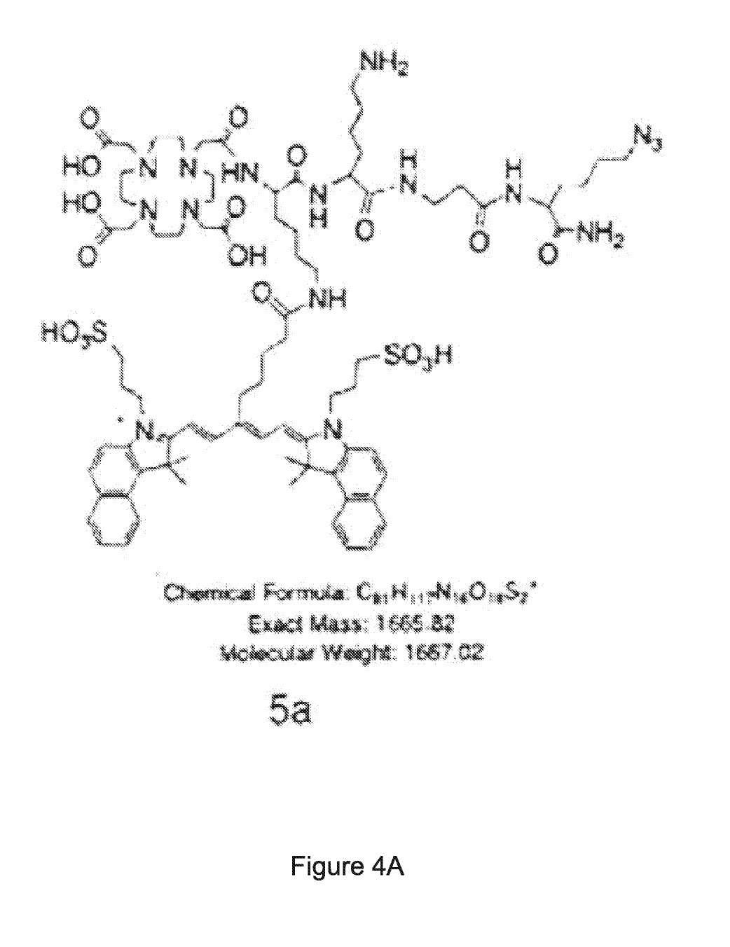

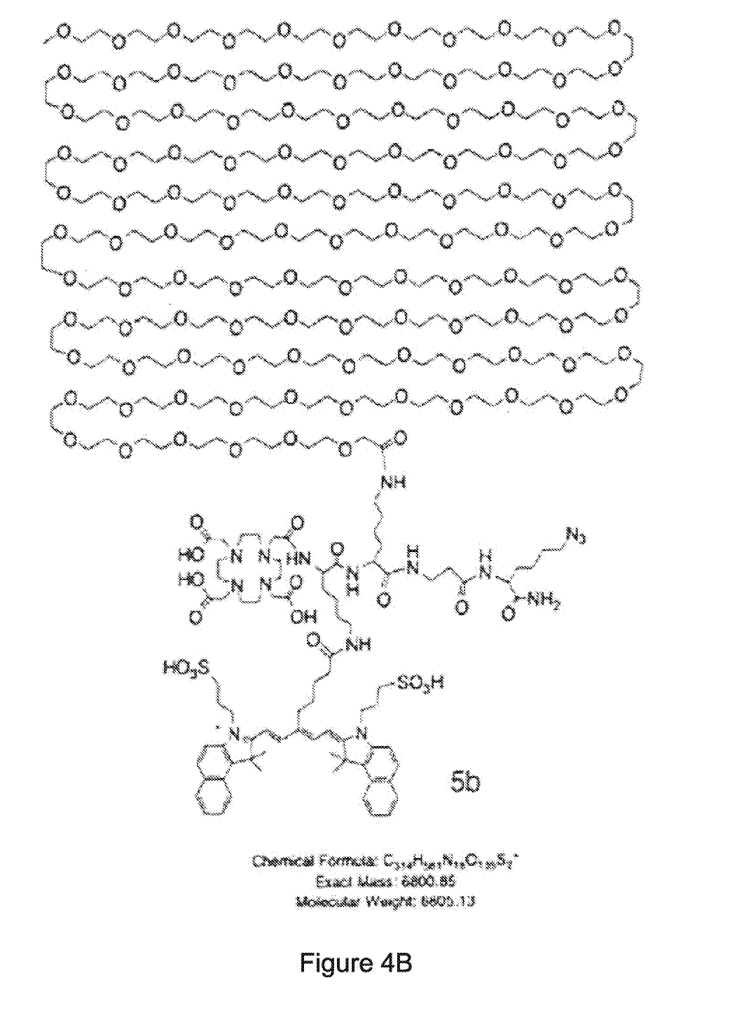

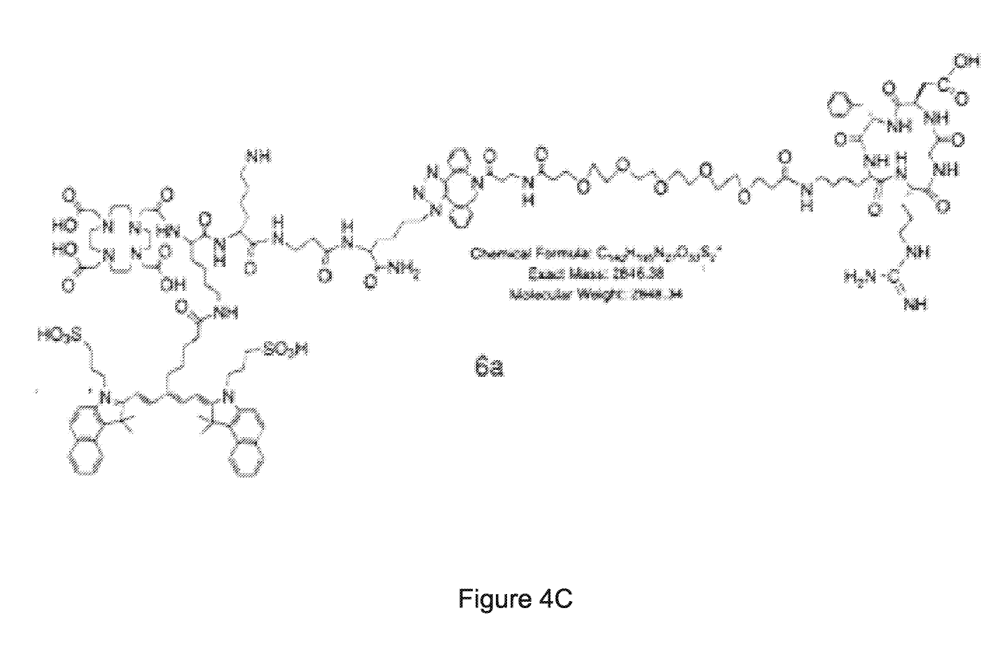

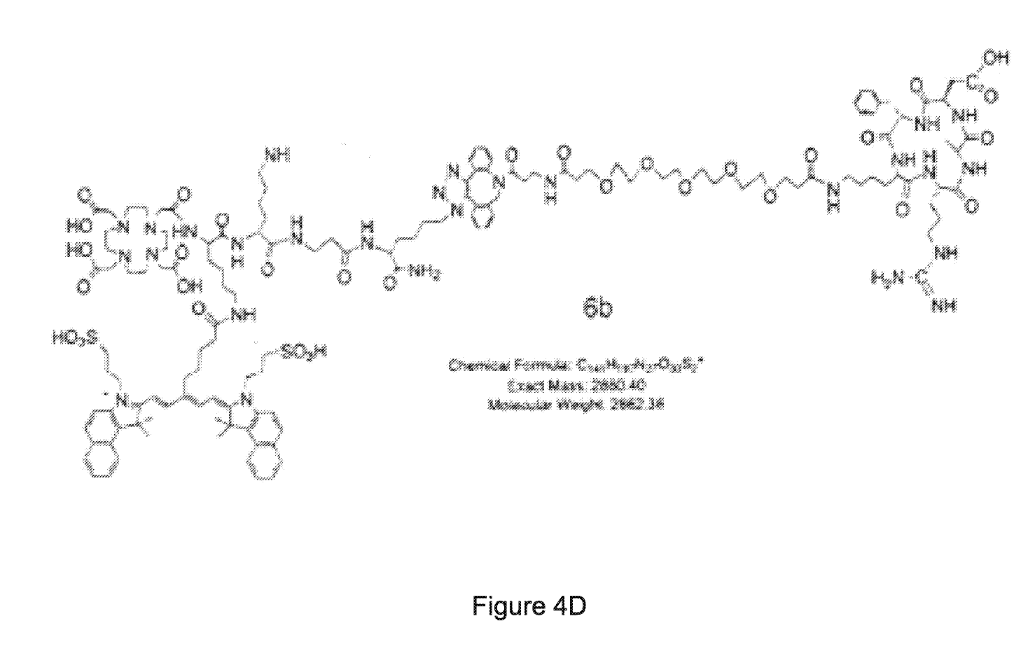

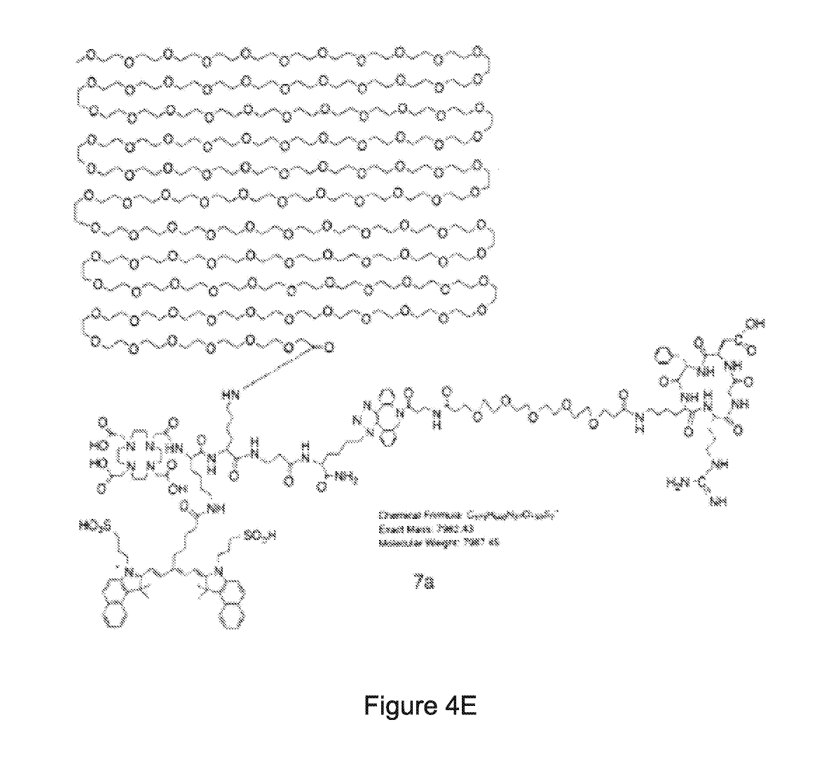

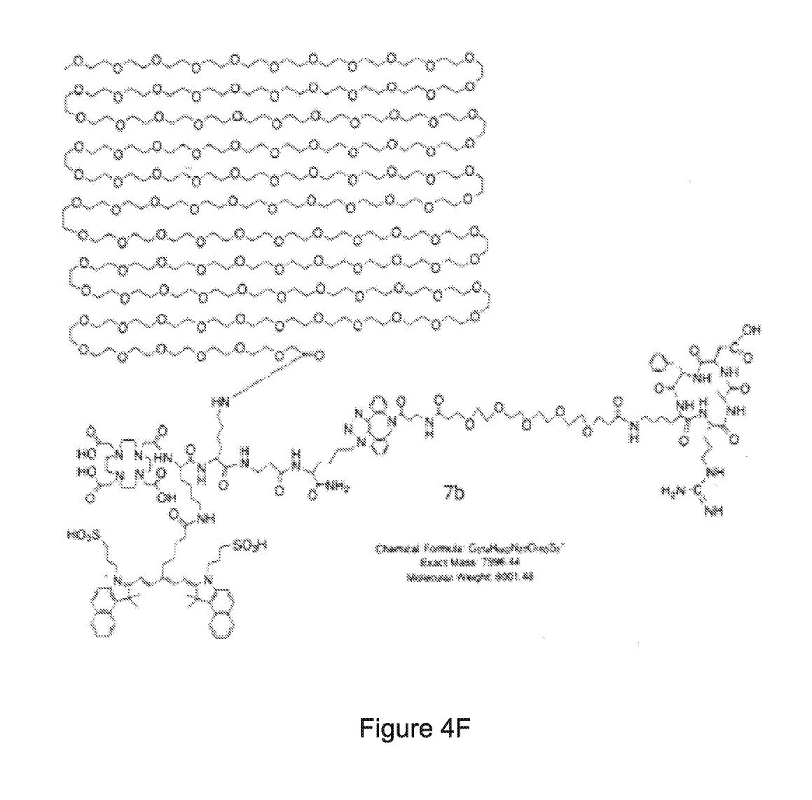

A strategy that was used to synthesize trifunctional RGD and RAD is shown in FIG. 1. In FIG. 1(a), a multifunctional reagent module was first synthesized and attached to a linker-targeting vehicle module via a copperless click reaction, to yield a multifunctional probe. PEGylation was conferred by the 5 kDa PEG at the F3 position. FIG. 1(b) shows the synthesis of linker-targeting vehicles bearing RGD (arginine, glycine, aspartate) or RAD (arginine, alanine, aspartate) peptides. FIG. 1(c) shows the synthesis of multifunctional probes with functional groups of F1=DOTA, F2=CyAL5.5 fluorochrome, and F3=5 kDa PEG. The reaction conditions were: a: 1) NH₂NH₂, DMF; 2) CyAL5.5 Acid/PyBOP/DMF/DIPEA; 3) TFA; b: PEG-5K-NHS, DMSO; c: 5a, cRGD-PEG4-DBCO (3a) or RAD-PEG4-DBCO (3b), DMSO; d: PEG-5K-NHS. Complete structures are given in FIGS. 4A to 4F.

### Materials and Methods

Protected L-amino acids, PyBOP and Rink Amide MBHA resin were from Novabiochem (EMD Biosciences). Other special chemicals were from other sources: DOTA(CO₂Bu^{t})₃ (Macrocyclics), mPEG-NHS ester (5 kDa) (Creative PEGworks), Fmoc-Lys(N₃)—OH (AnaSpec), and DBCO-PEG₄-NHS (Click Chemistry Tools). The fluorescent dye CyAL5.5 was synthesized as described in United States Patent Application Publication No. 2011/0286933. CyAL5.5 is also available from Molecular Targeting Technologies, Inc., West Chester, Pa., USA. All the other solvents and chemicals were from Sigma-Aldrich. Molecular weights were obtained by MS-ESI Micromass (Waters) and MALDI-TOF analyses at the Tufts University Core Facility. RP-HPLC (Varian ProStar detector and delivery modules) employed an eluant A (0.1% TFA/water) and eluant B (0.1% TFA in 9.9% water in acetonitrile). RGD and RAD peptides were cRGDfK and cRADfK from Peptides International.

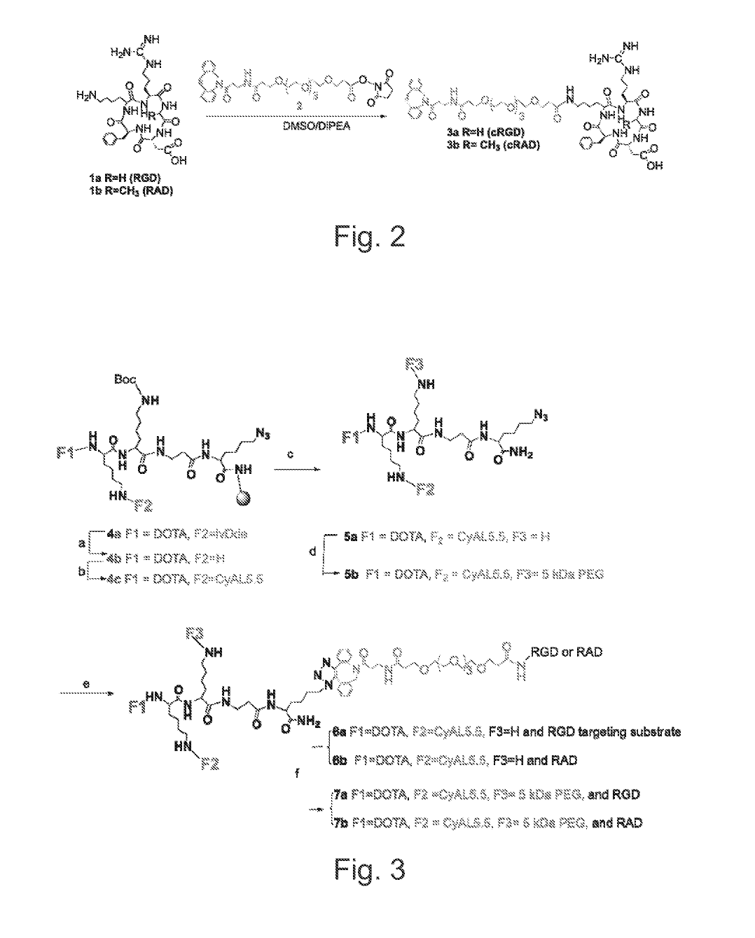

FIG. 2 shows the synthesis of linker RGD (or RAD) targeting vehicles. For (3a): A stock solution of DBCO-PEG₄-NHS ester (2) (containing 7.5 mg, 10.8 μmol) in anhydrous DMSO was added to the solution of the RGD peptide, cRGDfK (3a) (5.6 mg, 9.28 μmol from Peptides International) in anhydrous DMSO (0.4 ml). After DiPEA (9 μl) was added, the mixture was incubated at room temperature overnight. After diluted with buffer A, the mixture was purified by HPLC with gradient of 20% B-100% B in 15 minutes, then back to 20% B in 5 minutes and isocratic for 5 minutes; flow: 12 ml/min; λmax: 226 nm; column: Higgins Analytical Inc. Clipeus C18, 10 μm, 250×20 mm, P/N: CS-2520-C181, S/N: 186532. A white powder (5a) was obtained. Yield: >90% C₅₉H₇₉N₁₁O₁₅, MW: 1182.32, MS: Cal. 1181.58, Observed: 1182.11. For (3b), the procedure was followed 3a by using the RAD peptide cRADfK with similar results. C₆₀H₈₁N₁₁O₁₅, MW: 1196.35, MS: Cal. 1195.59, Observed: 1196.30.

FIG. 3 shows the synthesis of trifunctional probes. a: NH₂NH₂, DMF; b: CyAL5.5 Acid, PyBOP, DMF, DIPEA; c: TFA; d: PEG-5K-NHS, DMSO; e: 5a, cRGD-PEG4-DBCO (3a) or RAD-PEG4-DBCO (3b), DMSO; f: PEG-5K-NHS.

### Synthesis of 5a

### Comparative Example

The DOTA(CO₂—Bu^{t})₃-Lys(ivDde)-Lys(Boc)-β-Ala-Lys(N₃) peptide (4a) was manually synthesized on Rink Amide MBHA resin (0.15 mmol) with an Fmoc/t-Bu strategy using a polypropylene 5-mL disposable syringe fitted with a sintered frit. Coupling reactions employed 2 equiv. (relative to resin) of N-α-Fmoc-protected amino acid activated in situ with 2 equiv. of PyBOP and 4 equiv. of DiPEA in DMF (10 mL/g resin) for 1-2 hrs. Coupling efficiency was assessed with picrylsulfonic acid. N-α-Fmoc groups were removed with a piperidine/DMF solution (1:4) for 4×10 minutes (10 mL/g resin). The coupling of DOTA was overnight with same equivalent of other reagents. After intermediate (4b) was obtained by N-ε-ivDde group removal with 2% hydrazine in DMF for 5 minutes (10 mL/g resin), the attachment of CyAL5.5 (for intermediate 4c) was carried out on the solid phase for overnight by using CyAL5.5 acid (2 equiv.) under the in situ activation of PyBOP (2 equiv.) and DiPEA (8 equiv.). Intermediate DOTA-Lys(CyAL5.5)-Lys(NH₂)-β-Ala-Lys(N₃) (5a) was released from the solid support with TFA/H₂O/TIS/EDT 88:2:5:5 (twice, 4 h, 20 mL/g resin). After the solvent was evaporated, the residue was precipitated and triturated with cold ether. A blue solid could be obtained by centrifuge. The solid was purified further by preparative HPLC with a gradient of 20%-80% B in 15 minutes, back to 20% B in 3 minutes, and isocratic for 3 minutes; λmax: 670 nm; flow: 21 ml/min; column: Higgins Analytical Inc., Clipeus C18 10 μm, 250×20 mm. A blue powder of compound (5a—see FIG. 4A) was obtained after lyophilization with a yield of 40%. C₈₁H₁₁₇N₁₆O₁₈S₂⁺; MW: 1667.02; MS: cal. 1665.82. found (m/z): 1666.2 and 833.8.

### Synthesis of 5b

To a solution of DOTA-Lys(CyAL5.5)-Lys(NH₂)-β-Ala-Lys(N₃)—NH₂ (5a) (1.0 mg, 0.6 μmol) in anhydrous DMSO (0.4 ml), was added the solution of m-PEG-5K-NHS (13.8 mg, 2.76 μmol) in anhydrous DMSO (0.5 ml). After DiPEA (10 μL) was added, the reaction mixture was incubated at room temperature for 3 days. The mixture was diluted by acetonitrile and water (0.1% TFA, 1:1 v/v) and purified by HPLC with a gradient of 20%-100% B in 20 minutes, then back to 20% B in 5 minutes and isocratic for 5 minutes; flow: 5 ml/min; λmax: 670 nm; Varian Pursuit XRs 5 C18, 250×10 mm column, P/N: A6000250X100, S/N: 1007962. A blue powder (5b—see FIG. 4B) was obtained after lyophilization. Yield: >90%. Mass was in a wide range from 6200 to 7100 due to PEG.

### Synthesis of unPEGylated RGD and RAD probes, 6a, 6b

### Comparative Examples

For 6a, a mixture of the solution of DOTA-Lys(CyAL5.5)-Lys(NH₂)-β-Ala-Lys(N₃)—NH₂ (5a) (3.2 mg, 1.92 μmol) in DMSO (0.4 ml) with the solution of DBCO-PEG₄-cRGD (3a) (2.5 mg, 2.11 μmol) in DMSO (0.4 ml) was incubated for 2 hours at room temperature. The product was purified by HPLC with a gradient of 20%-100% B in 20 minutes, then back to 20% B in 5 minutes and isocratic for 5 minutes; flow: 5 ml/min; λmax: 670 nm; column: Varian Pursuit XRs 5 C18, 250×10 mm, P/N: A6000250X100, S/N: 1007962. A blue powder (6a—see FIG. 4C) was obtained. Yield: ˜99%. C₁₄₀H₁₉₆N₂₇O₃₃S₂⁺, MW: 2849.34, MS: cal. 2847.39. found: 2848.29. For (6b—see FIG. 4D), the procedure was followed 6a by using 3b with similar results. C₁₄₁H₁₉₈N₂₇O₃₃S₂⁺, MW: 2863.37, MS: cal. 2861.41. found: 2862.22

### Synthesis of Multifunctional PEGylated RGD and RAD Probes, 7a and 7b

To a solution of DOTA-Lys(CyAL5.5)-Lys(NH₂)-β-Ala-Lys(N₃-DBCO-PEG₄-cRGD)-NH₂ (6a) (1.54 mg, 0.54 μmol) in DMSO (0.9 ml), was added the solution of m-PEG-5K-NHS (18 mg, 3.6 μmol). After DiPEA (10 μL) was added, the reaction mixture was incubated at room temperature for 3 days. The mixture was diluted by acetonitrile and water (0.1% TFA, 1:1 v/v) and purified by HPLC purification with a gradient of 20%-100% B in 20 minutes, then back to 20% B in 5 minutes and isocratic for 5 minutes; flow: 5 ml/min. λmax: 670 nm; column: Varian Pursuit XRs 5 C18, 250×10 mm, P/N: A6000250X100, S/N: 1007962. A blue powder (7a—see FIG. 4E) was obtained. Yield: >90%. Mass was observed in a wide range from 7300 to 8300 due to PEG. For (7b—see FIG. 4F), the procedure was followed 7a by using 6b with similar results. Masses ranged from 7300 to 8300 Da due to PEG polydispersity.

The structures of compounds 5a to 7b are given in FIGS. 4A to 4F. The most prominent species in the polydisperse 5 kDa PEG is n=115, and is shown for 5b, 7a and 7b.

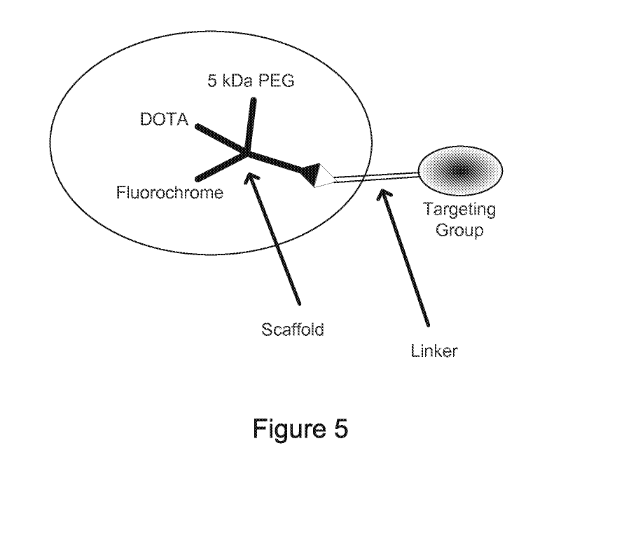

FIG. 5 shows how the tetrapeptide scaffold, bearing the PEG and fluorochrome is attached to a “targeting moiety” or “targeting group” which binds to a molecular target expressed by a cell within the tumor. Looking at FIG. 5, the targeting group is the smaller oval. PEG shields the fluorochrome, providing a diffuse cloud (the larger oval in FIG. 5). A linker is shown between the scaffold, bearing the PEG and fluorochrome, and the targeting group.

### Synthesis of ¹¹¹Indium Labeled Probes RGD and RAD Probes, 9a and 9b

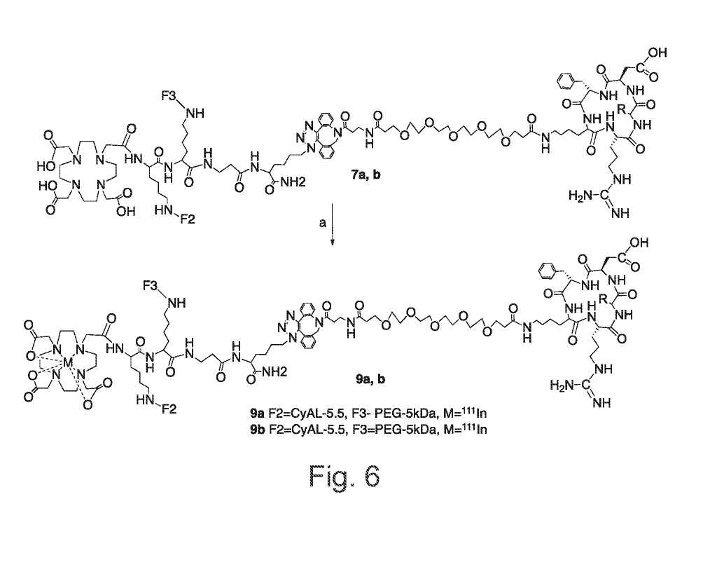

The synthesis of 111-indium labeled probes RGD and RAD probes (9a,9b) is shown in FIG. 6. For the ¹¹¹In labeling of 7a or 7b. a: ¹¹¹InCl₃, 1M HEPES, pH 5, 70° C. To the solution of 7a or 7b (20 nmoles), reconstituted with 1M HEPES buffer (pH 5.0) (0.7 ml) in a 5 ml conic react vial, was added the solution of ¹¹¹InCl₃ in 0.05N HCl (150 μl, 333 MBq, 9 mCi). After 45 minutes at 70° C., the mixture was cooled in a water bath for 2 minutes. A stock solution of EDTA (70 mM, 50 μl) was added and the solution was allowed to stay at room temperature for 15 minutes. After the mixture was diluted with ammonium acetate buffer (0.5 ml, 1M, pH 6), and loaded onto a C18 cartridge preconditioned by ethanol (0.5 ml, 0.1% acetic acid) and water (1 ml, 0.1% acetic acid) sequentially. The labeled tracers (9a,9b) were purified by washing the cartridge with water (1 ml, 0.1% acetic acid) and collected by eluting with acetonitrile (200 μl, 0.1% TFA). The acetonitrile and TFA were removed by co-evaporation with ethanol (3×200 μl) together with Ar flow and reconstituted with 0.9% saline for injection. Radioactive products were identified by their co-chromatography with the corresponding nonradioactive indium labeled compounds. Radiochemical yield (RCY) was 50-70%.

Probe Volume Determinations:

Size (volume) was determined by FPLC using an ÄKTA Purifier 10 and Superdex™ 75 10/300 GL column (GE Healthcare Lifesciences) with a running buffer of 0.05 M sodium phosphate, 0.15 M NaCl (0.1% Tween, pH 7.2) and flow rate of 0.5 ml/min. The protein standards (Gel Filtration Calibration Kit LMW, code no. 28-4038-41, GE Healthcare) (0.3 mg/ml, mixture of Aprotinin, Ribonuclease A, Ovalbumin, and Conalbumin) and Blue Dextran 2000 were used. To obtain volumes, Mr (apparent molecular weight based on size exclusion retention) was plotted versus Kav. Kav=(Ve−Vo)/(Vt−Ve), Vt=total volume, Ve=elution volume, Vo=void volume.

BT-20 Transfection for GFP Expression:

Day 1, BT-20 cells were planted into 24-well plate at 750,000 cells/well in culture medium (EMEM with 10% FBS). Day 2, the old culture medium was replaced with new culture medium containing lenti-virus (1×10⁸ particles/mL) and protamine sulphate (American Pharmaceutical Partners, Los Angelus, Calif.; 10 mg/mL; 1:500 of total volume of medium). Day 3, after 16 hours of transfection, medium was changed to the culture medium (EMEM with 10% FBS). 8 hours later, green cells could be seen via fluorescence microscope.

BT-20 Tumor Model:

All animal experiments were approved by the Institutional Review Committee of Massachusetts General Hospital. Female nude mice (25-30 g; 6-8 weeks old; nu/nu; Cox 7, Massachusetts General Hospital, Boston, Mass.) were anesthetized with isoflurane/O₂. Tumor cell implantation was performed both sides around the shoulder. 200 μl of cell suspension containing 10⁶ cells in Matrigel (BD) was injected subcutaneously. Tumor cells were inoculated for 7 to 10 days.

Biodistribution of Indium-111 Labeled 7a or 7b:

150 μl of Indium-111 (with 300 μCi) labeled compounds 7a or 7b were injected to BT-20 tumor-bearing animals intravenously. 24 hours later, animals were sacrificed, organs, such as, tumors, blood, liver, spleen, stomach, kidneys, small intestine, lung, heart, tail, fat, and muscle, were collected. The radioactivities of those organs were measured by gamma counter (Perkin Elmer, Wizard² 2480).

SPECT/CT Imaging:

150 μl of Indium-111 (with 300 μCi radioactivity) labeled compound 7a was injected to BT-20 tumor-bearing animals intravenously. 24 hours later, SPECT/CT images were taken with a triple modality microPET-SPECT-CT imaging device (Triumph, GE Healthcare).

### Example 2

### Diffusion Molecular Retention (DMR) Technique

The molecularly targeted delivery of toxic “payloads” to tumors is limited by low tumor blood flow, capillary permeability barriers, high interstitial pressure, and kidney, liver and spleen uptake. We demonstrated a technique termed Diffusion Molecular Retention (DMR) that comprises peritumorally injecting a fluorescent peptide probe, visualizing probe extensive probe diffusion through the interstitium by fluorescence, and obtaining retention if the probe encounters a molecular target. DMR employs peptide probes that by virtue of their PEGylation achieve a volume of 25 kDa, and therefore have a slow vascular uptake, as well as an absence of non-specific binding to components of the interstitium. To demonstrate DMR, a trifunctional RGD probe bearing a DOTA, a 5 kDa PEG and fluorochrome was synthesized and interstitial diffusion visualized by surface fluorescence. A control RAD probe was not retained, indicating retention of the RGD probe was due to integrin binding. With DMR and a [¹¹¹In] RGD probe, SPECT-CT indicated a highly specific tumor uptake, with tumor concentrations of 390% ID/gm (percentage injected dose per gram) compared to only 4% ID/gm by intravenous administration. DMR could be used to visualize tumor margins by intraoperative fluorescence or to deliver high doses of radiotoxic metals to invasive but non-metastatic tumors. Given the difficulties encountered with high efficiency molecular delivery of diagnostic or therapeutic “payloads” to solid tumors by intravenous administration, the DMR technique might be evaluated in a variety of settings.

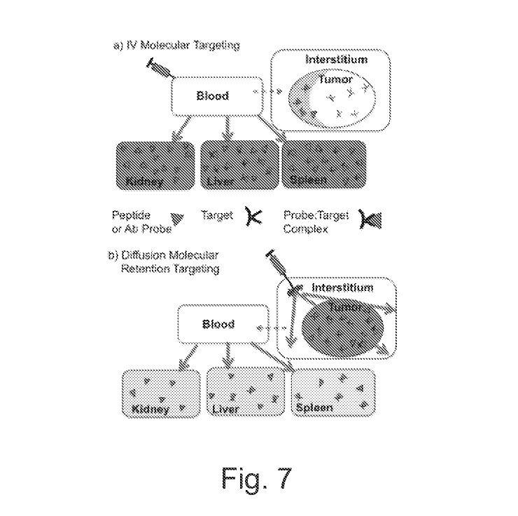

FIG. 7a summarizes the results of an intravenous injection of a peptide or antibody probe binding a molecular target expressed on a tumor and normal tissues where high probe concentrations occur in the liver, kidney or other organs (and resulting in dose-limiting toxicities). Normal organ accumulation can be a target mediated (e.g. RGD probes binding integrins expressed in normal tissues) or a non-target mediated (non-specific) accumulation.

Here we present an alternative to intravenous administration called Diffusion Molecular Retention (DMR), that increases the fraction of an injected probe retained by a tumor due to molecular interactions. DMR comprises (see FIG. 7b) of a peritumoral (PT) injection of a PEGylated fluorochrome and chelate bearing probe, observing probe diffusion through the interstitium by fluorescence, and obtaining probe retention if the probe encounters a molecular target to which it binds. Potential applications of DMR include the delivery of NIR fluorochromes to tumors for intraoperative margin delineation and the delivery of radioisotopes (e.g. toxic, short range alpha emitters) to tumors for radiotherapy.

To illustrate DMR, we synthesized multifunctional integrin binding RGD and control RAD probes as in Example 1 above. Integrin specificity of the RGD probe cells or tissues was taken as the difference in binding of RGD and RAD probes, which differ in a 15 dalton methyl group out of total mass of about 8000 daltons, see Table A below. Synthesis employed a multifunctional reagent module consisting of a peptide scaffold, and DOTA, CyAL5.5 fluorochrome and 5 kDa PEG functional groups. The multifunctional reagent module in peptide notation can be written as (DOTA)Lys(CyAL5.5)-Lys(5 kDa PEG)-βala-Lys(N₃). The reagent module was the reacted with RGD or RAD peptides bearing a short PEG spacer and terminal dibenzylcyclooctyne (DBCO) group, using a copperless click reaction. DOTA was used to chelate ¹¹¹In³⁺ for SPECT-CT and quantitative biodistribution studies, while the CyAL5.5 fluorochrome was used to visualize diffusion from the peritumoral injection site by surface fluorochrome. The 5 kDa PEG endowed the RGD or RAD probes with a volume of 25 kDa, similar to that of small proteins (e.g. Fv=12 kDa, scFV=25 kDa), since PEG's assume far greater volumes in solution than suggested by their molecular weight. The 5 kDa PEG creates a diffuse cloud that blocks RGD/integrin mediated interactions. Physical properties of RGD and RAD probes are summarized in Table A.

TABLE A
Physical Properties of Integrin Targeted and Control Probes

| | Target | MW | MS Obs. | Equiv. Vol. |
|---|---|---|---|---|
| | Peptide | (Da) | (Da) | (kDa) |
| RGD Probe | RGD | 7987.4 | 7980 (peak) | 25 |
| RAD probe | RAD | 8001.4 | 8000 (peak) | 25 |

The diffusion and elimination of the non-integrin binding RAD probe after an intramuscular administration in the front extremity of a nude mouse was visualized. Using surface fluorescence, the probe rapidly diffused through the extremity and shoulder of the mouse, with vascular uptake and renal elimination evident from bladder fluorescence at 4 hours post injection. By 24 hours post injection, detectable fluorescence was not found, indicating clearance from the injection site.

The diffusion and molecular retention required of the DMR technique with a tumor bearing model were investigated. We peritumorally injected the RGD probe into an animal bearing two GFP expressing BT-20 breast carcinomas and monitored tumor GFP fluorescence and probe fluorescence as a function of time after injection. The non-integrin binding RAD probe was injected into a second animal also bearing two tumors. Overlaying purple probe fluorescence over green GFP yields white. The BT-20 cell line binds RGD peptides and antibodies to the αᵥβ₃ integrin. Both probes rapidly diffused from their injection sites, surrounding the tumor within 10 minutes of the injection. The RGD probe was retained by the tumor while the RAD probe was cleared by 24 hours post injection. Tumor surface fluorescence from both probes was quantified by the use of solution standards. With the RAD probe tumor fluorescence at 24 hours post injection was not observed, indicating that the fluorescence retained at 24 hours with RGD was due to molecular interactions with RGD binding integrins.

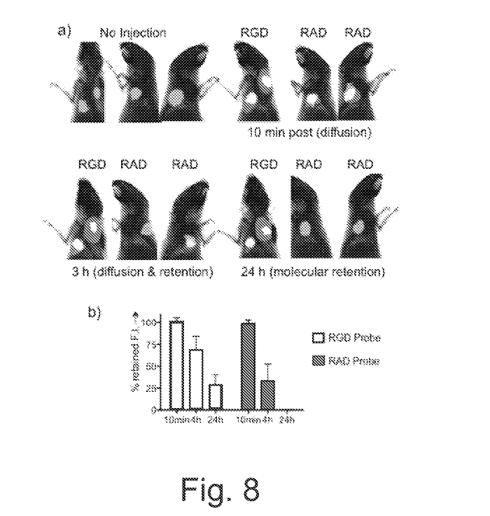

FIG. 8 shows tumor targeting by DMR by using the GFP expressing BT-20 breast carcinoma xenograft by surface fluorescence. In FIG. 8a), two animals bearing two tumors were PT injected with the RGD probe or RAD probe as indicated and surface fluorescence images were obtained. With the RAD injected animal, tumors were more sagittal so two views of the same animal are provided. Green equaled GFP. Purple equaled probe. White equaled green+purple overlay. The RGD probe diffused around the tumor and is retained while the RAD probe was eliminated. In FIG. 8b), quantitation of tumor surface fluorescence after injections of the RGD or RAD probes as above. Surface fluorescence was quantified through the use of standard solutions. Only the RGD probe was retained by the tumor. n=4, values are mean±1 SEM.

To compare the DMR and intravenous (IV) methods, surface fluorescence images of tumor GFP and RGD probe fluorescence were obtained with skin removed, and overlays from the two signals obtained. With both DMR and IV administration, probe fluorescence extended beyond tumor GFP margins to a stromal area beyond the tumor. However, tumor fluorescence was far higher with DMR than IV injection, even though dose was far lower (50 pmoles/mouse by DMR versus 2000 pmoles/mouse by IV).

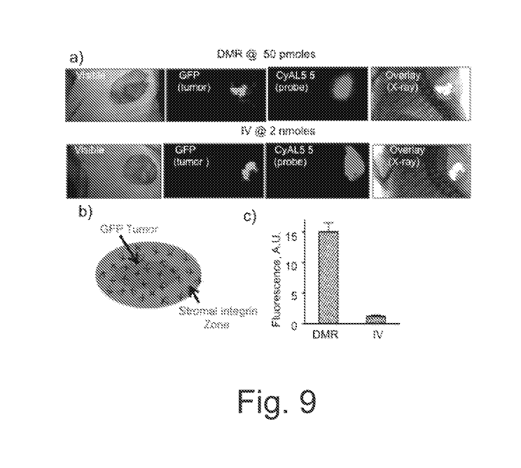

FIG. 9 shows the efficiency of tumor targeting by DMR or IV methods. In FIG. 9a), skin covering GFP-BT-20 tumor was removed. Shown are visible GFP fluorescence, probe (CyAL5.5) fluorescence, and the overlay of GFP and probe fluorescence plus an X-ray image. Green GFP plus purple CyAL5.5 fluorescence yielded a white overlaid image. In FIG. 9b), with DMR or IV, probe fluorescence included a stromal zone of integrin binding surrounding the tumor was seen. In FIG. 9c), a comparison of tumor surface fluorescence by DMR versus the IV methods is shown. Doses were 50 pmoles (DMR) and 2 nmoles (IV).

SPECT-CT images were obtained with the ¹¹¹In labeled RGD probe by the DMR and IV methods. With IV administration, radioactivity was predominant in the liver, kidney and small intestine, with a small tumor radioactivity seen at 2 hours post injection. Radioactivity in the lower abdomen was from the stomach and small intestine based dissection studies. With a single DMR administration, radioactivity was concentrated in the tumor at 2 hours post injection and exclusively in the tumor at 24 hours post injection.

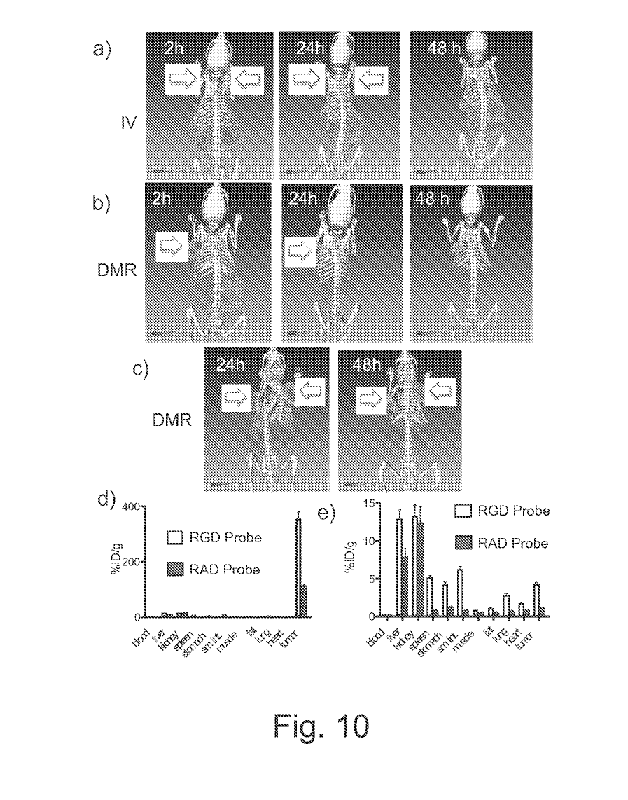

FIG. 10 shows SPECT/CT images after DMR and IV injections with the ¹¹¹In RGD probe using the BT-20 tumor model. Images after single injections of the ¹¹¹In-RGD probe by the IV (FIG. 10a) or DMR (FIG. 10b) methods are shown. Radioactivity is shown with a green to red color scale, while CT bone density is yellow. White arrows show single or double tumors. In FIG. 10c), images after dual DMR injections are shown at 24 hours and 48 hours post injection. In FIG. 10d), tissue radioactivity concentrations obtained with the ¹¹¹In-RGD and ¹¹¹In-RAD probes by DMR. In FIG. 10e), radioactivity per organ with the ¹¹¹In-RGD and ¹¹¹In-RAD probes by IV is shown. Radioactivity was 0.3 mCi per injection IV and single and dual DMR injection.

Tissue concentrations were then obtained with the IV and DMR methods using an ¹¹¹In labeled RGD probe and an ¹¹¹In RAD probe. With the RGD probe tumor radioactivity was 390% ID/gm by DMR versus 4% ID/gm by IV administration. With both DMR and IV, tumor radioactivity was highly dependent on molecular interactions with integrins. Markedly higher tumor probe concentrations with DMR relative to IV was seen with both fluorescence and radioactive measurements. Tumor fluorescence was 15.0 au (absorbance units) with DMR compared to 1.5 au with IV.

DMR employs a peritumoral administration, followed by visualizing the high interstitial diffusion that follows with fluorescence, to deliver high levels of an RGD probe to integrins expressed by the BT-20 tumor. To obtain the extensive interstitial diffusion needed for molecular targeting with the peritumoral administration, two conditions must be obtained. First, transport from the interstitial space to the vascular compartment (blood) must be slow, providing the time needed for extensive interstitial diffusion. The 5 kDa PEG increased probe volume to that of a small protein, and conferred a highly hydrophilic character on the probe, both of which slow the rate of interstitial to vascular compartment transport. Second, the probe must not adhere to components of the interstitium, so that complete clearance from the injection site is obtained in the absence of molecular interactions. Probes had blood half-lives (blood fluorescence after IV injection) of 10.7 minutes and underwent predominant renal elimination.

A variety of minimally invasive injection or local injection techniques might permit peritumoral injection with tumors in a variety of anatomical settings. Local injection techniques are used for sentinel lymph node determination, treating benign prostatic hyperplasia, treating urinary incontinence, and for stem cell delivery.

The use of fluorescence to observe probe diffusion after a PT injection is a key feature of DMR. This may permit a determination of probe diffusion with human tumors, which will be larger and which will occur in a wider variety of anatomical locations than those seen with our mouse xenografts. Multiple PT injection sites and modest volumes (0.1 to 0.5 mL) may be employed to enable probes to diffuse through larger and more varied human tumors. Our DMR method in the mouse employed 50 pmoles of probe (3.0 ng as RGD peptide) corresponding to 8.4 μg of peptide for a 70 kg human. With human tumors multiple injection sites with 10 μg of peptide per site might be used with minimal systemic chemotoxicity from the targeting peptide. Dosage could vary from 0.001 μg/kg to 10 μg/kg.

The modular synthetic strategy (see Example 1) used to obtain DMR probes allows two types of substitutions. First, using this principle we have shown that a variety of fluorochromes, chelates and PEG functional groups can be attached to scaffold peptides for subsequent reactions with a targeting peptide. Second, using the multifunctional reagent employed here, other receptor targeted peptides bearing a single amino functional group might be used as targeting vehicles. A personalized selection of targeting peptide might be based on a histochemical method of determining peptide/receptor in tumor section.

We have demonstrated the use of DMR as attractive drug delivery alternative to IV injection. These principles are peritumoral injection, visualization of the extensive interstitial diffusion by fluorescence, and molecular retention. A second important goal was to employ a modular synthetic approach that may permit peptides binding various molecular targets, and delivering a wide variety of “payloads”, to be used. We do suggest that an attractive class of applications for DMR lies with the molecularly targeted delivery of fluorochromes to invasive tumors that are operable only with a high functional loss. Here the greatly reduced amounts of probe used with DMR may reduce costs, particularly prominent with the use of NIR fluorochromes, and reduce the risks of systemic chemotoxicity. With human tissue microarrays, an RGD peptide bound ductal carcinomas (22 of 25) but not normal breast (2 of 10), suggesting a PEGylated, NIR probe bearing an RGD targeting peptide might be useful in this setting. See, Montet et al., (2006) Enzyme-based visualization of receptor-ligand binding in tissues, Lab Invest. 86(5):517-25. A second attractive class of DMR applications is the delivery of therapeutic radioisotopes to invasive, pre-metastatic tumors. Here DMR offers the delivery of high radiation doses to tumors and greatly reduced radiation burdens to normal organs. The DOTA functional group can chelate a range of metals for SPECT or PET (¹¹¹I, ⁶⁸Ga) or radiotherapy (e.g. ²¹³Bi, ¹⁷⁷Lu, ⁹⁰Y, or ²²⁵Ac. DMR maybe particularly well suited to the delivery of alpha particle emitters, with their high toxicity and short range of action.

Given the frustrating difficulties encountered with efficient molecular delivery of toxic “payloads” to solid tumors by the IV administration, the DMR technique may find use in selected settings.

### Example 3

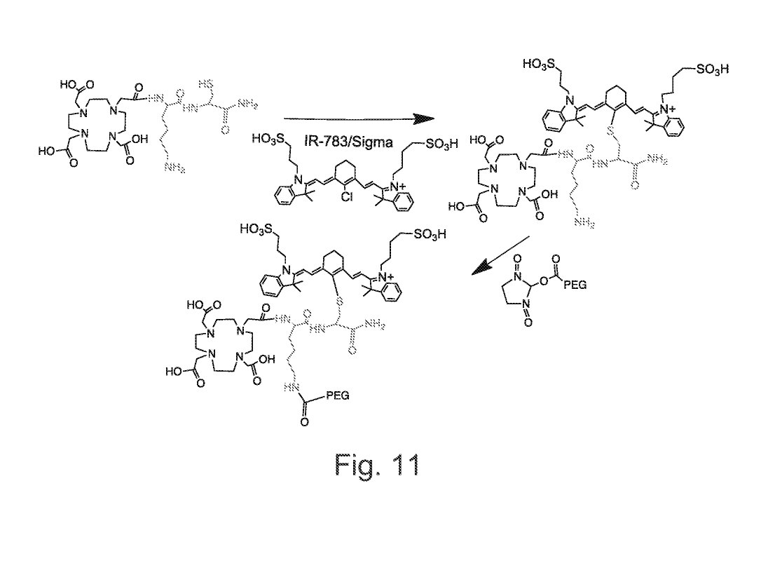

A general synthesis of PEG-like fluorochromes on a dipeptide scaffold is shown in the FIG. 11, where a Lys-Cys dipeptide is employed. The DOTA-Lys-Cys-NH₂ peptide was made by solid phase synthesis as described in Garanger (2010) “Divergent oriented synthesis for the design of reagents for protein conjugation,” J Comb Chem. 12(1):57-64. The DOTA-dipeptide was reacted with IR-783, and then with an NHS (N-hydroxysuccinimide) ester of a PEG (MW's variable) to yield the compounds shown in Table 3B.

The reaction of DOTA-Lys-Cys-NH₂ with the thiol reactive IR-783 followed procedures in Garanger above or with slight modifications. A solvent of dry DMSO was employed with 1.5 to 2.5 equivalents of IR-783 per equivalent of dipeptide and DIEPA added (2-4 equivalents DIEPA per equivalent of dipeptide). Typically DOTA-dipeptide amounts were 2-20 μmoles in 0.2 to 2 mL of DMSO. Reaction was overnight at room temperature. The reaction mixture was purified by reverse phase HPLC, which separated unreacted IR-783 from DOTA-Lys-Cys(IR-783)-NH₂. Further purification or determination of volume was by size exclusion FPLC.

DOTA-Lys-Cys(IR783)-NH₂ is then dissolved in DMSO, with 2 equivalents of an NHS ester of PEG and 6 equivalents of DIPEA. The reaction was at room temperature for 5 days. Product was purified by reverse phase HPLC separation. Product molecular weight was by matrix-assisted laser desorption ionization time-of-flight mass spectrometry (MALDI-TOF MS), with product molecular volume by size exclusion FPLC using globular protein standards (GE Healthcare life science AKTApurifier 10).

Using these procedures and PEG's of different molecular weights, PEG-like NIR fluorochromes of different molecular weights, volumes and pharmacokinetics were synthesized, with results summarized in Table 3B. Blood half-lives were determined by tail injection into mice and measuring serum fluorescence as a function of time. Data (fluorescence versus time) were fit to a single decay constant equation to yield blood half-lives. Human pharmacokinetics will be far slower than that of mice.

### Example 4

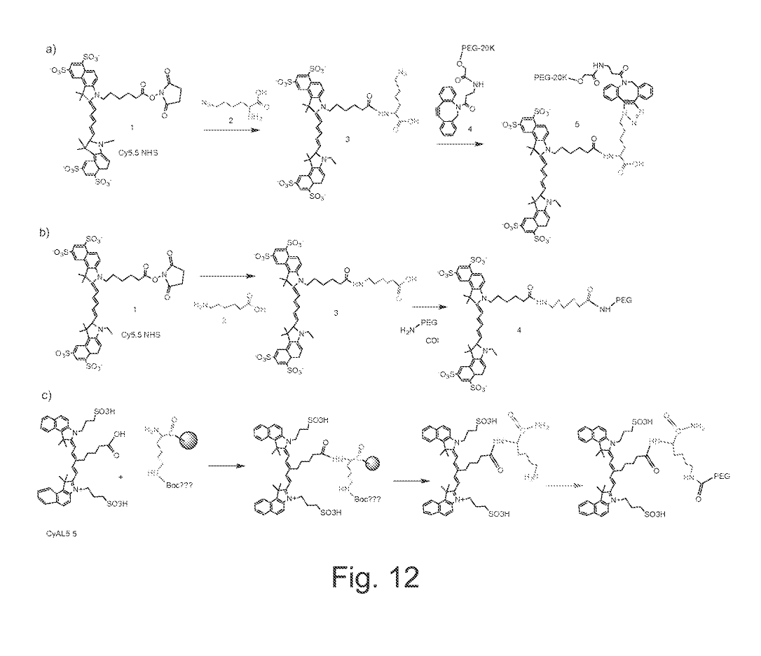

Three proposed, general methods (a, b, c) of reacting a PEG and a fluorochrome with an amino acid are shown in FIG. 12.

For FIG. 12 a), a mixture of Cy5.5-NHS ester (1, 1 mmol), 6-azidolysine (2, 1 mmol), and DIPEA (5 mmol) will be incubated under room temperature in anhydrous DMSO for overnight. TFA (5 mmol) will be added to neutralize the DIPEA. After the mixture is diluted with water:acetonitrile (1:1, v/v), the product (3) can be isolated by HPLC.

The powder of 3 (1 mmol) and 20KDa-PEG-DBCO (4, 1 mmol) will be mixed and incubated together in DMSO for overnight. After the mixture will be diluted with water:acetonitrile (1:1, v/v), the product (5) will be isolated by HPLC. Additional purification might be obtained by FPLC SEC purification.

For FIG. 12 b), a mixture of Cy5.5-NHS ester (1, 1 mmol), 6-aminohexanoic acid (2, 1 mmol), and DIPEA (5 mmol) will be incubated under room temperature in anhydrous DMSO for overnight. TFA (5 mmol) will be added to neutralize the DIPEA. After the mixture will be diluted with water:acetonitrile (1:1, v/v), the product (3) will be isolated by HPLC.

The powder of 3 (1 mmol) and PEG-amine (1 mmol) will be mixed and incubated together in the presence of EDC (2 mmol) in DMSO for overnight. After the mixture is diluted with water:acetonitrile (1:1, v/v), the product (4) will be isolated by HPLC. More pure product might be obtained by FPLC SEC purification.

For FIG. 12 c), Fmoc-Lys(Boc)-OH will be attached to Rink Amide MBHA resin (0.15 mmol) with an Fmoc/t-Bu strategy using a polypropylene 5-mL disposable syringe fitted with a sintered frit. Coupling reactions will employ 2 equiv. (relative to resin) of N-α-Fmoc-protected lysine activated in situ with 2 equiv. of PyBOP and 4 eq. of DIPEA in DMF (10 mL/g resin) for 1-2 hrs. Coupling efficiency will be assessed with trinitrobenzylsulfonyl. N-α-Fmoc groups can be removed with a piperidine/DMF solution (1:4) for 4×10 min (10 mL/g resin). The coupling of CyAL5.5 will be overnight with same equivalents as with the other reagents. The deprotected CyAL5.5-Ly(NH₂)—NH₂ intermediate will be released from the solid support with TFA/H₂O/TIS/EDT 88:2:5:5 (twice, 4 h, 20 mL/g resin). After the solvent will be evaporated, the residue will be precipitated and triturated with ether. The blue solid will be obtained by centrifugation. The solid will be purified further by preparative HPLC with a C18 column. The PEGylation will be carried out by the incubation for 2 days in anhydrous DMSO in the presence of PEG-NHS (1.5 eq.) and DIPEA (2 eq.). The mixture will be diluted with water:acetonitrile (1:1, v/v), and the product will be isolated by HPLC.

Table 3C below provides a summary of other proposed Example 4 compounds.

### Example 5

### Direct Linkage of PEG and Fluorochrome

Table 3D below provides a summary of the proposed Example 5 compounds.

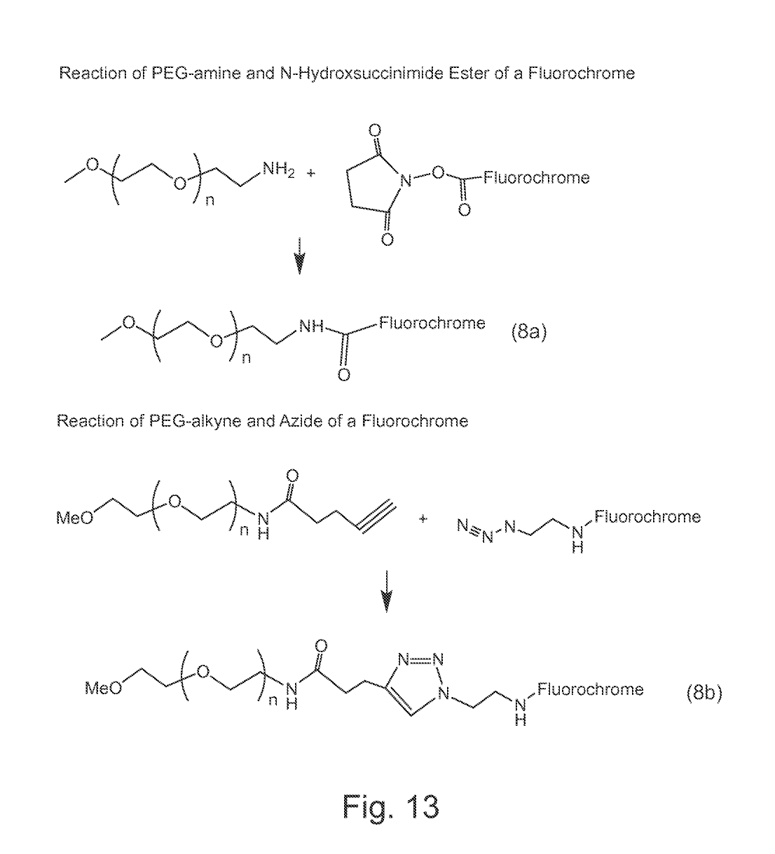

FIG. 13 gives two general strategies for reacting fluorochromes and PEG's. One could synthesize a fluorescent compound (8a) having a direct linkage of PEG and fluorochrome by reacting an amine reactive methoxy-PEG (mPEG−M_{W}=5000) available from Nanocs, Boston, Mass., USA with a Cy5 fluorochrome NHS ester (available from Lumiprobe). Still referring to FIG. 13, one could synthesize another fluorescent compound (8b) having a direct linkage of PEG and fluorochrome by reacting an alkyne reactive methoxy-PEG (M_{W}=5000) available from IRIS Biotech GmbH, Marktredwitz, Germany with a Cy5.5 fluorochrome azide (available from Lumiprobe).

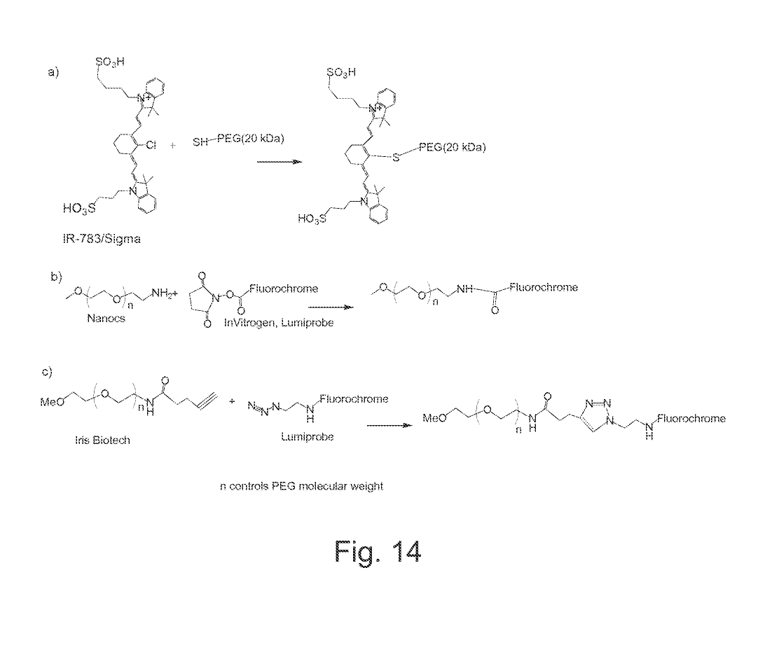

Three proposed, general methods of directly reacting a PEG with a fluorochrome are shown in FIG. 14. For FIG. 14(a), a mixture of PEG-SH (1 mmol), IR-783 (3 mmol), and DIPEA (3 mmol) will be incubated under room temperature in anhydrous DMSO with the protection by Ar for 3 days. TFA (3 mmol) will be added to neutralize the DIPEA. After dilution with water:acetonitrile (1:1, v/v), the product will be isolated by HPLC. A more pure product will be obtained by FPLC SEC purification, if necessary. For FIG. 14 (b), a mixture of a fluorochrome-NHS ester (2 mmol), a PEG-NH₂ (1 mmol), with DIPEA (2 mmol) will be incubated in anhydrous DMSO for overnight at room temperature. TFA (2 mmol) will be added to neutralize the DIPEA. After the mixture will be diluted with water, acetonitrile (1:1, v/v) and the product will be isolated by HPLC. For FIG. 14(c), to a solution of PEG-alkyne (3 mmol) and fluorochrome azide (3 mmol) in water, will be added to sodium ascorbate (0.3 mmol, 300 μL of freshly prepared 1 M solution in water), followed by copper(II) sulfate pentahydrate (7.5 mg, 0.03 mmol, in 100 μL of water). The mixture will be stirred vigorously overnight and purified with HPLC with C18 column.

Tables 3A to 3D provide a summary of passively targeted PEG-like fluorochromes relevant to the intravenous diagnostic method.

TABLE 3A
PEG-like Fluorochromes for Passive Targeting Intravenous Diagnostic Method
PEG and Fluorochromes attached to tetrapeptides

| | | | | Wavelength | |
|---|---|---|---|---|---|
| | | Molecular | | Absorption/ | |
| | Mw, | Volume | Quantum | Emission | |
| Compound | (kDa) | (kDa) | Yield | (nm.) | Comment |
| CyAl5.5 | | | 0.08 | 675/695 | Fluorochrome |
| Fluorochrome | | | | | Reference |
| DOTA-Lys(CyAL5.5)-Lys-βAla- | 1.667 | <1 | 0.061 | 675/695 | No PEG |
| Lys(N₃)-NH₂ | | | | | Control |
| (See 5a of Example 1) | | | | | |
| DOTA-Lys(CyAL5.5)- | 6.805 | <1 | 0.17 | 675/695 | Passive |
| Lys(5 kDa PEG)- βAla - | | | | | Targeting, |
| Lys(N₃)-NH₂ | | | | | Fluorochrome |
| (See 5b of Example 1) | | | | | PEG |
| | | | | | Shielded |
| DOTA-Lys(CyAL5.5)-Lys- βAla - | 2.848 | <1 | 0.082 | 675/695 | No PEG |
| Lys(sPEG-RGD), | | | | | Control |
| (See 6a of Example 1) | | | | | |
| DOTA-Lys(CyAL5.5)-Lys- βala - | 2.862 | <1 | 0.081 | 675/695 | No PEG |
| Lys(sPEG-RAD), | | | | | Control |
| (See 6b of Example 1) | | | | | |
| DOTA-Lys(CyAL5.5)- | 8.002 | 20 | 0.20 | 675/695 | Passive |
| Lys(5 kDa PEG)- βala - | | | | | Targeting, |
| Lys(sPEG-RAD), | | | | | Fluorochrome |
| (See 7b of Example 1) | | | | | PEG |
| | | | | | Shielded |

TABLE 3B
PEG-like Fluorochromes for Passive Targeting Intravenous Diagnostic Method
PEG and Fluorochromes attached to dipeptides

| | | Molecular | | Wavelength | Serum half- |
|---|---|---|---|---|---|
| | | Volume | | Absorption/ | life, |
| | Mw, | FPLC | Quantum | Emission | min |
| Compound | (kDa) | (kDa) | Yield | (nm.) | (mouse, IV) |
| ICG | 0.775 | | 0.041 | | |
| Fluorochrome | | | | | |
| (Fluorochrome reference) | | | | | |
| IR 783 | 0.748 | | 0.043 | 782/795 | |
| Fluorochrome | | | | | |
| (Fluorochrome reference) | | | | | |
| DOTA-Lys-Cys(IR783)-NH₂ | 1.329 | 0.600 | 0.053 | 792/807 | |
| (Example 3) | | | | | |
| (No PEG Control) | | | | | |
| DOTA-Lys(PEG 2 kDa)- | 3.341 | 11.2 | 0.15 | 789/810 | 1.4 |
| Cys(IR783)-NH₂ | | | | | |
| (Example 3) | | | | | |
| DOTA-Lys(PEG 5 kDa)- | 6.290 | 35.2 | 0.17 | 789/810 | 13.1 |
| Cys(IR783)-NH₂ | | | | | |
| (Example 3) | | | | | |
| (Passive Targeting, | | | | | |
| Fluorochrome PEG Shielded) | | | | | |
| DOTA-Lys(PEG 10 kDa)- | 11.405 | 100.7 | 0.17 | 789/810 | |
| Cys(IR783)-NH₂ | | | | | |
| (Example 3) | | | | | |
| DOTA-Lys(PEG 20 kDa)- | 21.283 | 262.5 | 0.17 | 789/810 | 17.6 |
| Cys(IR783)-NH₂ | | | | | |
| (Example 3) | | | | | |
| DOTA-Lys(PEG 40 kDa)- | 44.995 | 690 | 0.18 | 789/809 | |
| Cys(IR783)-NH₂ | | | | | |
| (Example 3) | | | | | |
| (Passive Targeting, | | | | | |
| PEG Shield) | | | | | |

TABLE 3C
PEG-like Fluorochromes for Passive Targeting Intravenous Diagnostic Method
PEG and Fluorochromes attached to Amino Acids

| | | | | Wavelength | |
|---|---|---|---|---|---|
| | | Molecular | | Absorption/ | |
| | Mw, | Volume | Quantum | Emission | |
| Compound | (kDa) | (kDa) | Yield | (nm.) | Comment |
| DOTA-Lys(PEG 40 kDa)- | 43.581 | 690 | 0.60 | 490/510 | Passive, |
| Cys(Fl)-NH₂ | | | | | Fluorochrome |
| (Example 4) | | | | | PEG Shielded |
| NH₂-Cys(IR-783)-OH | | <2 | <0.10 | 780/800 | No PEG |
| (Example 4) | | | | | Control |
| 5 kDa PEG-Cys | | >20 | >10 | 780/800 | Passive, |
| (IR-783)-OH | | | | | Fluorochrome |
| (Example 4) | | | | | PEG Shielded |

Fl = fluorescein

TABLE 3D
PEG-like Fluorochromes for Passive Targeting Intravenous
Diagnostic Method Direct linkage PEG and Fluorochrome

| | | | | | |
|---|---|---|---|---|---|
| | | | | Wavelength | |
| | | Molecular | | Absorption/ | |
| | Mw, | Volume | Quantum | Emission | |
| Compound | (kDa) | (kDa) | Yield | (nm.) | Comment |

| | | | | |
|---|---|---|---|---|
| 8a in FIG. 13 | 2-40 | 20-400 | >0.10 | NIR Range |
| (Example 5) | | | | |
| 8b in FIG. 13 | 2-40 | 20-400 | >0.10 | NIR Range |
| (Example 5) | | | | |

Table 4 provides a summary of actively targeted PEG-like fluorochromes relevant to the Diffusion Molecular Retention (DMR) method.

TABLE 4
PEG-like Fluorochromes Active Targeting DMR method

| | | | | Wavelength | |
|---|---|---|---|---|---|
| | | Molecular | | Absorption/ | |
| | Mw, | Volume | Quantum | Emission | |
| Compound | (kDa) | (kDa) | Yield | (nm.) | Comment |
| DOTA-Lys(CyAL5.5)- | 7.987 | 20-30 | 0.20 | 675/695 | Active |
| Lys(5 kDa PEG)- βAla - | | | | | Targeting, |
| Lys(sPEG-RGD), | | | | | Fluorochrome |
| (See 7a of Example 1) | | | | | PEG Shielded |
| DOTA-Lys(CyAL5.5)- | 8.002 | 20-30 | 0.20 | 675/695 | Targeting |
| Lys(5 kDa PEG)- βAla - | | | | | control, |
| Lys(sPEG-RAD), | | | | | Fluorochrome |
| (See 7b of Example 1) | | | | | PEG Shielded |

### Example 6

### Synthesis of PEG-Like Nanoprobes

In this Example, we prepared multimodal, pharmacokinetically and optically tunable nanomaterials.

### Materials and Methods

Protected L-amino acids, PyBOP and Rink Amide MBHA resin were from Novabiochem (EMD Biosciences). Other special chemicals were from other sources: DOTA(CO₂Bu^{t})₃ (Macrocyclics), mPEG-NHS ester (2-30 kDa from Creative PEGworks; 40 kDa from NOF corporation, Japan). The fluorescent dye IR-783 was purchased from Sigma-Aldrich, fluorescein-5-maleimide was from Thermo Scientific, and Cy3-maleimide was from Lumiprobe. All the other solvents and chemicals were from Sigma-Aldrich.

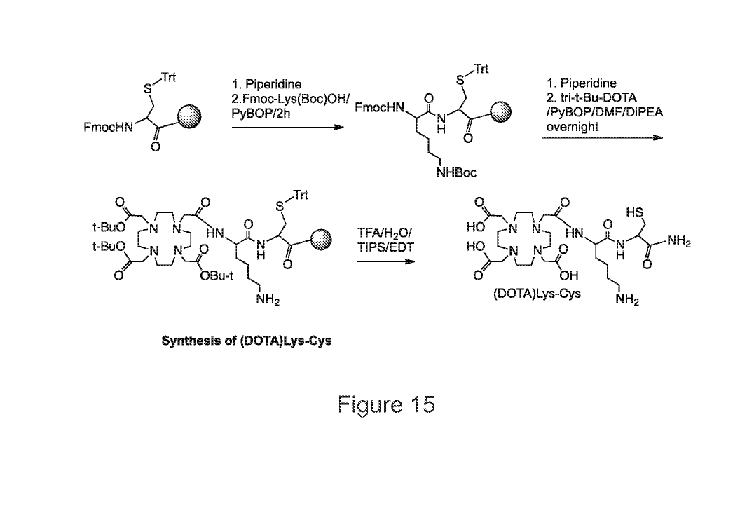

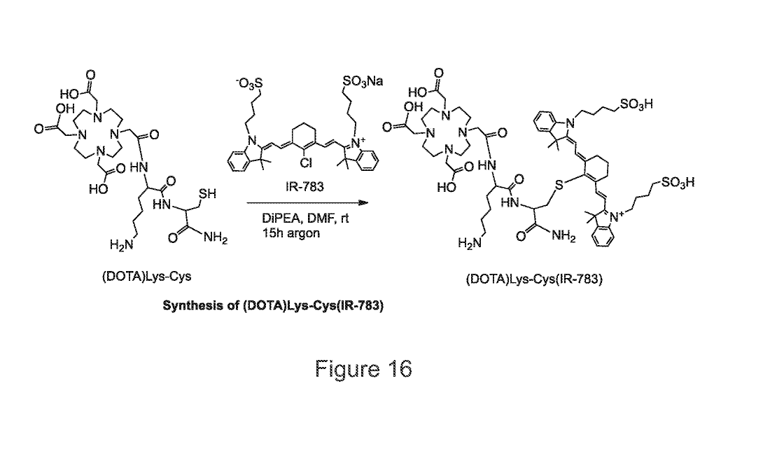

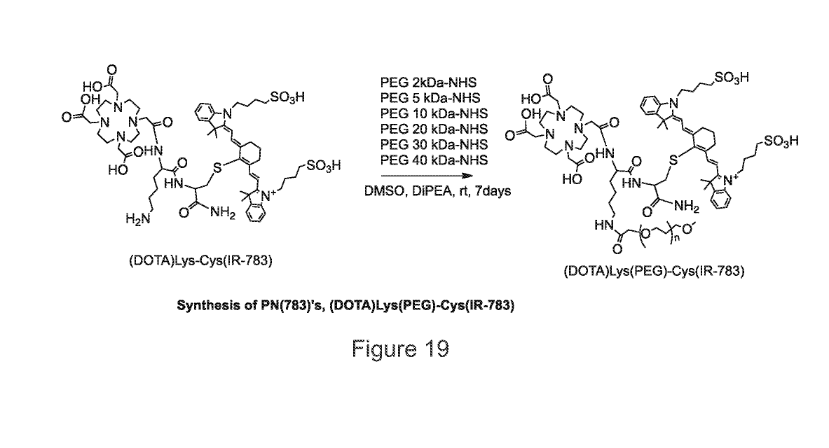

The synthesis of PEG-like nanoprobes (PN's) involves three steps: (i) synthesis of the (DOTA)Lys-Cys peptide (see FIG. 15), (ii) reaction of thiol reactive fluorochrome to the cysteine thiol (see FIGS. 16, 17, and 18) and, (iii) reaction of an NHS-ester of PEG with variable molecular weight to the lysine side chain (see FIGS. 19,20 and 21).

(i) Synthesis of the (DOTA)Lys-Cys Peptide (See FIG. 15):

The DOTA(CO₂Bu^{t})₃-Lys(Boc)-Cys(Trt) peptide was manually synthesized on Rink Amide MBHA resin (0.15 mmol) with an Fmoc/t-Bu strategy using a polypropylene 5 mL disposable syringe fitted with a sintered frit. Coupling reactions employed 2 equiv. (relative to resin) of Fmoc-protected amino acid activated in situ with 2 equiv. of PyBOP and 4 equiv. of DiPEA in DMF (10 mL/g resin) for 1-2 hrs. Coupling efficiency was assessed with picrylsulfonic acid. Fmoc groups were removed with a piperidine/DMF solution (1:4) for 4×10 min (10 mL/g resin). The coupling of DOTA was overnight with same equivalent of other reagents. (DOTA)Lys-Cys was released from the solid support with TFA/H₂O/TIS/EDT 88:2:5:5 (twice, 4 h, 20 mL/g resin). The residue was precipitated and triturated with cold ether. A white solid could be obtained by centrifuge. The solid was purified further by HPLC with column: Higgins Analytical Inc., Clipeus C18 10 μm, 250×20 mm; gradient: 20%-100% B (0.1% TFA and 9.9% water in acetonitrile) in 15 minutes, back to 20% B in 5 minutes, and isocratic for 5 minutes. A white powder of compound (DOTA)Lys-Cys was obtained after lyophilization with a yield of 40%. For (DOTA)Lys-Cys, theoretical MW=634.75. found MW (M+1)=635.57.

(ii) Synthesis of (DOTA)Lys-Cys(FL) Peptides where FL can be IR-783, Cy3 or Fluorescein. See FIGS. 16-18:

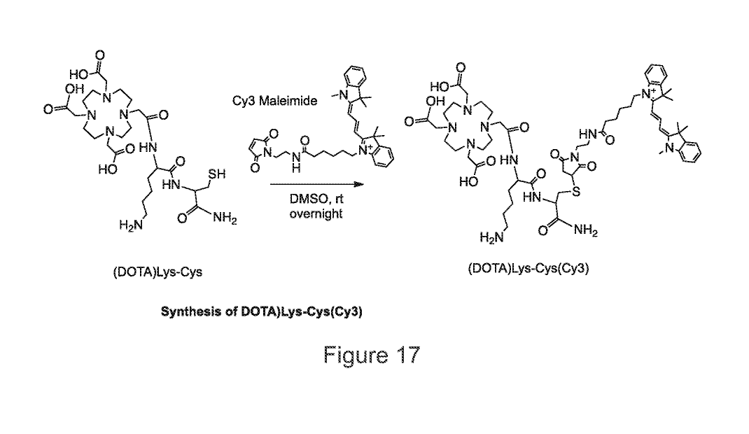

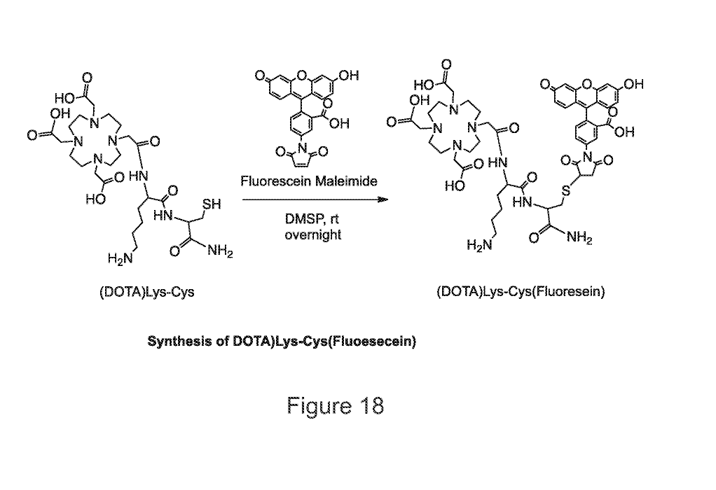

With all three fluorochromes, the molar ratio of (DOTA)Lys-Cys to fluorochrome 1:1.2. Reaction with IR783 was in DMF, under argon, at room temperature for 15 hours, with 6 equiv of DiPEA (see FIG. 16). The reaction with Cy3-maleimide (see FIG. 17) or Fluorescein-maleimide (see FIG. 18) was in DMSO at room temperature for overnight. Products were purified with reverse phase HPLC with a C18 column. The yield of each was around 45% with respect to the starting quantity of fluorochrome. For (DOTA)Lys-Cys(IR-783), theoretical MW=1325.6. found MW=1325.8. For (DOTA)Lys-Cys(Cy3), theoretical MW=1213.6. found MW=1213.8. For (DOTA)Lys-Cys(Fluorescein), theoretical MW=1061.4. found (M+1)=1062.6.

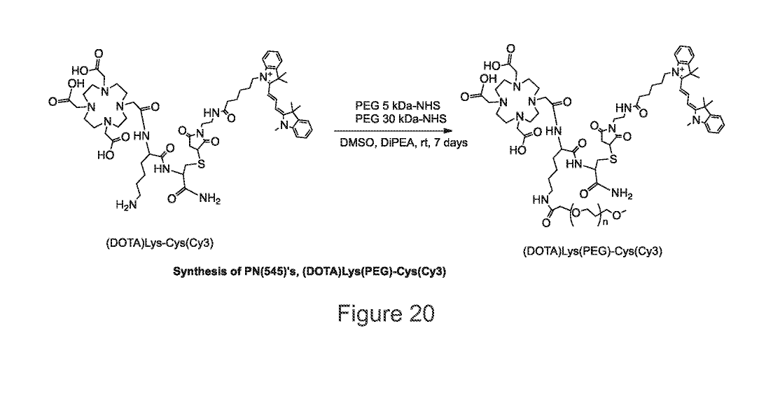

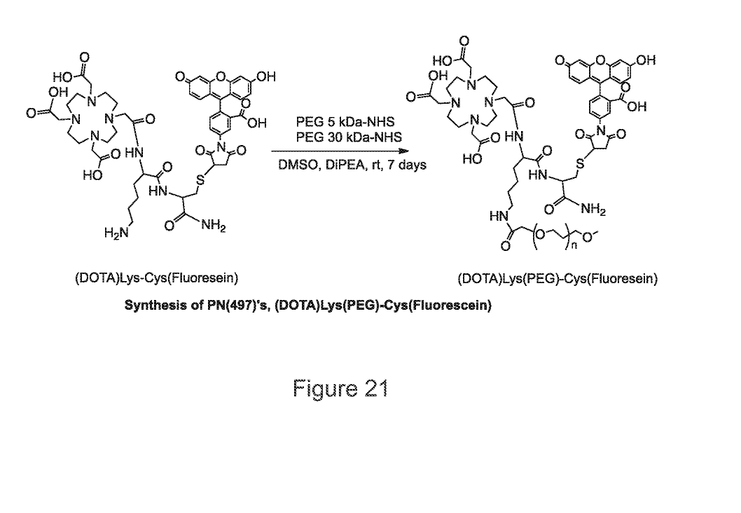

(iii) Reaction of the (DOTA)Lys-Cys(FL) with NHS Esters of PEG, See FIGS. 19-21.

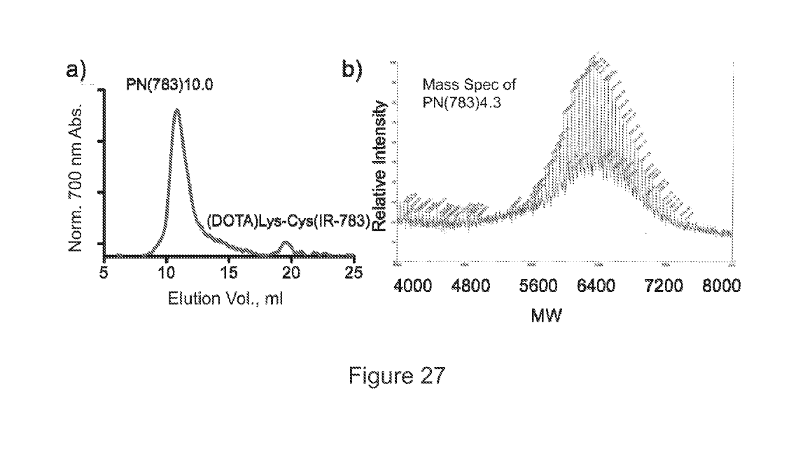

To a solution of (DOTA)Lys-Cys(fluorochrome) in anhydrous DMSO, was added the solution of PEG-NHS in anhydrous DMSO. The molar ratio of (DOTA)Lys-Cys(fluorochrome) and PEG-NHS was 1:2. After about 6 equiv. of DiPEA was added, the reaction mixture was incubated at room temperature for 7 days. Purification was first by a reverse phase HPLC (C18 column) with gradients as described in (i) to remove low molecular weight impurities from the synthesis and to obtain an exchange to an aqueous solvent. After lyophilization a second purification was by FPLC, which removed traces of non-PEGylated peptides, see FIG. 27. PN's were concentrated and desalted with a C18 cartridge (Sep-Pak cartridge, Waters, Milford, Mass., USA), eluting with acetonitrile and drying by lyophilization. The yield based on starting (DOTA)Lys-Cys(FL), was about 50%.

PN and Peptide Characterization:

The mass spec of low molecular weights (MW) were obtained by MS-ESI Micromass (Waters) and high MW molecules were determined through MALDI-TOF analyses at the Tufts University Core Facility. RP-HPLC (Varian ProStar detector and delivery modules) employed an eluant A (0.1% TFA/water) and eluant B (0.1% TFA and 9.9% water in acetonitrile). Probe size (volume) was determined by FPLC using an ÄKTA Purifier 10 and Superdex™ 200 10/300GL column (GE Healthcare) with a running buffer of 0.05 M sodium phosphate, 0.15 M NaCl (0.1% Tween, pH 7.2) and flow rate of 0.8 ml/min. Standards (GE Healthcare) were Ferritin, Ribonuclease A, Carbonic Anhydrase, and Conalbumin and Blue Dextran 2000. To obtain probe volumes, Mr (apparent molecular weight based on size exclusion retention) was plotted versus Kav. Kav=(Ve−Vo)/(Vt−Vo), Vt=total volume, Ve=elution volume, Vo=void volume.

Purity of Materials Made:

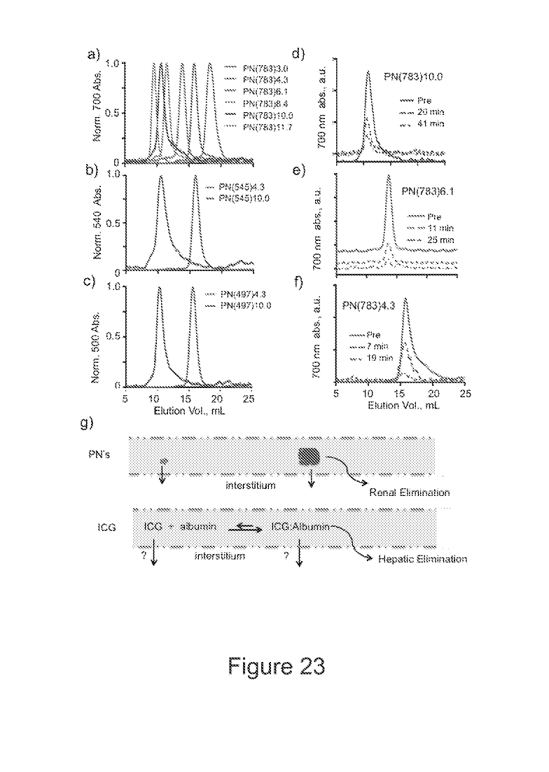

The four peptides used (see Table 5) were characterized by mass spectroscopy. The use of FPLC to remove low molecular weight peptide is shown in FIG. 27a. FPLC's of the purified PN's are shown in FIG. 23. The mass spec of PN(783)4.3, the peptide (DOTA)Lys(PEG 5 kDa)-Cys(IR-783), is shown in FIG. 27b.

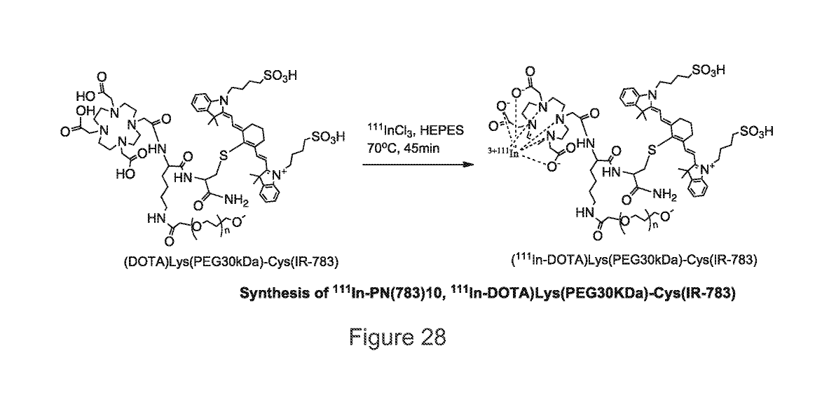

Radiolabelling of PN(783)10.0. See FIG. 28:

¹¹¹InCl₃ (9.43 mCi) (Nordion, Canada) was diluted with HCl (50 μl, 0.05N) into a total volume of 80 μl and was transferred into a conic reaction vial which contained PN(783)/10.0 (20 nmol) in HEPES buffer (1 M, pH 5, 1 ml). The reaction vial was incubated on a preheated heating blot under 70° C. for 45 minutes while it was shaken every 10 minutes. Then the vial was cooled down to room temperature in ice water for 5 minutes. EDTA (70 mM, 100 μl, 7 mmol) was added and well mixed. The solution was stayed at room temperature for 15 minutes. After the solution was diluted with water (0.1% TFA) (1:10 v/v), the compound was loaded on a C18 cartridge preconditioned with ethanol (1 ml, 0.1% TFA) and water (3 ml, 0.1% TFA) (Strata-X 33u, Polymeric reverse Phase Phenomenex, 30 mg/3 ml, 8B-S100-TBL). The cartridge was washed with water (Millipore, 0.1% HOAc) (2 ml) and purged by air with a syringe. The labeled compound was collected by eluting with acetonitrile (0.1% TFA) (0.4 ml) into a new reaction vial. The acetonitrile and TFA were removed by evaporation under N₂ flow. The final product (4.06 mCi) was reconstituted with PBS buffer for mouse injection. The radioactive product was confirmed to be free of low molecular weight forms of indium by HPLC with cold internal standard with C18 column. (Gradient: 10% B to 100% B in 20 minutes, back to 10% in 5 minutes, and isocratic for 5 minutes; Abs: 783 nm; flow: 5 ml/min; Column: Higgins Analytical Inc. Proto 300 C18 5 μm, 250×10 mm, P/N: CS-2520-C185). RCY: 43%; specific activity: 0.4 Ci/μmole.

Quantum Yield:

Quantum yields were determined as described in Demas et al., “Measurement of photoluminescence quantum yields. Review”, Journal of Physical Chemistry 75, 991-1024 (1971), and Shao et al., “Facile Synthesis of Monofunctional Pentamethine Carbocyanine Fluorophores. Dyes and pigments: an international journal 90, 119-122 (2011). For IR-783 a reference quantum yield of 0.043 was used (see Li et al., “Synthesis and characterization of glucosamine-bound near-infrared probes for optical imaging”, Organic letters 8, 3623-3626, 2006); for fluorescein a reference quantum yield was 0.18 (see Sjoback et al., “Absorption and florescence properties of fluorescein”, Spectrohimica Acta Part A 51, L7-L21, 1995). For Cy3 a reference quantum yield of 0.31 was used (Luminprobe Inc). For PN(783)'s, excitation was at 730 nm and emission spectra were recorded from 765 nm to 870 nm in PBS and maximum emission used. For PN(545)'s, excitation was at 515 nm and emission spectra were recorded from 538 nm to 700 nm in PBS and maximum emission used. For PN(497)'s, excitation was at 450 nm and emission spectra were recorded from 475 nm to 620 nm in PBS and maximum emission used. Absorbance of each probe was adjusted less than 0.1. Measurements were made in triplicate and are expressed as mean±SD.

PN and Peptide Binding to Cells (Effect of PEGylation on NSB):

HT-29, a human colon carcinoma cell line, was from the American Tissue Culture Collection and maintained according to their instructions. Cells were seeded on 24-well plates at 5×10⁵ cells/well in culture medium (RPMI 1640 with 10% FBS) the day before the assay. The day of assay, medium was removed, wells rinsed twice with DPBS (+Ca, +Mg), and 100 μl of 2% FBS/DPBS (+Ca, +Mg) added. 100 μL of Nanoprobes (2 μM) in DPBS (+Ca, +Mg) was added to cells and incubated for 30 min at 37° C. (Probe concentrations were determined spectrophotometrically (783 nm, extinction coefficient of 314 471 cm⁻¹ M⁻¹ for IR-783; 497 nm, extinction coefficient of 68 000 cm⁻¹ M⁻¹ for Fluorescein; 545 nm, extinction coefficient of 150 000 cm⁻¹ M⁻¹ for Cy3). Cells were detached by Trypsin/EDTA and assayed for fluorescence by FACS (BD 7 laser LSR2 for nanoprobes with IR-783; BD 3 laser LSR2 for nanoprobes with Fluorescein or Cy3).

Circulating Form of PN's:

20 nmoles of PN(783)4.3, PN(783)6.1, or PN(783)10.0 was injected (IV, tail vein) into nude mice (female; 25-30 g; 6-8 weeks old; nu/nu). At the indicated time, 50 μl of blood was collected with microhematocrit capillary tube (Fisher Scientific) from the tail, and transferred to Eppendorf microcentrifuge tube with anticoagulant (EDTA) coating (Fisher Scientific). Tubes were centrifuged (5000 rpm for 5 minutes), and the supernatant was injected to the FPLC, a ÄKTA Purifier 10 with Superdex™ 200 10/300GL column.

PN Pharmacokinetics:

Groups of 5 nude mice (female; 25-30 g; 6-8 weeks old; nu/nu) were injected (tail vein, IV) with 10 nmole of PN(783)4.3 or PN(783)10.0. 50 μl of blood was collected from tail tip at the indicated times. The blood was processed as above, and diluted (25 μl plasma, 700 μl of PBS). Fluorescence was measured with Cary Eclipse Fluorescence Spectrophotometer, excitation at 765 nm and emission from 790 to 880 nm. The fluorescence intensity at 806 nm was plotted over time, and the data was fit with two-phase decay curve. The fast and slow distribution half-life was given by the two-phase decay fit with Graphpad Prism software.

Two Compartment Model:

From the two-phase decay fit, a biexponential equation for blood concentration as a function of time,

$$Cp = A \cdot {e}^{-} \text{?} + B \cdot {e}^{-} \text{?} , \\ \text{?} \text{indicates text missing or illegible when filed}$$

was obtained. By the relation of macro constants and micro constants,

$$k \text{?} - \frac{A β + B α}{A + B} , k \text{?} - \frac{αβ}{k \text{?}} , k \text{?} - α + β - k \text{?} - k \text{?} , \\ \text{?} \text{indicates text missing or illegible when filed}$$

micro constants k's can be obtained, and the half-life was calculated by

$$A 1 \text{/} 2 = 0.693 \text{/} k ,$$

as described in Rosenbaum, S. E. Basic Pharmacokinetics and Pharmacodynamics: An Integrated Textbook and Computer Simulations, (John Wiley and Sons, Hoboken, N.J., 2011). The curve for interstitium concentration vs. time was fit with MATLAB based on the curve of blood concentration vs. time.

Whole Animal Surface Fluorescence Imaging:

A Kodak FX multispectral imaging system was used (Carestream Molecular Imaging, Rochester, N.Y.). Excitation at multiple wavelengths (620, 650, 690, 710, 720, 730, 750 and 760 nm) with the emission at 830 nm was setup for IR-783 spectrum; Excitation at multiple wavelengths (420, 440, 460, 480, 510, 520, 530, and 540 nm) with the emission at 600 nm was setup for Cy3 spectrum; Excitation at multiple wavelengths (450, 470, 510, 520, 530, 540, 550, 570, and 590 nm) with the emission at 700 nm was setup for mCherry; with manufacturer's software to separate (unmix) the IR-783 spectrum, Cy3 spectrum, or mCherry spectrum from skin autofluorescence and chlorophyll fluorescence from food. X-ray images were taken after fluorescence images. Animals were anesthetized with 2% isoflurane with O₂ flow (2 I/min) during imaging.

Tumor Surface Fluorescence (Skin Removed):

The PN(783)10.0 or PN(545)10.0 (10 nmoles, 100 μL) was injected (IV, tail vein), the skin around tumor was removed at 48 hours post injection, with tumor visualized as mCherry fluorescence using the Kodak FX.

HT-29 or mCherry-HT-29 Tumor Model:

Female nude mice (25-30 g; 6-8 weeks old; nu/nu) were anesthetized with 2% isoflurane/O₂. HT-29 or mCherry-HT-29 cells were detached, pelleted and 200 μl of cell suspension containing 10⁶ cells in Matrigel (BD Bioscience) was injected subcutaneously into right and left shoulders. Tumors were allowed to grow 5-7 days before experiments.

SPECT/CT:

The imaging was performed by Triumph II multimodality imaging system (Gamma Medica Ideas, LLC) comprising XSPECT with four CZT (Cadmium Zink Telluride) detectors and X-O CT with CMOS detector. SPECT data of the ¹¹¹In-labeled compound was acquired for 60 minutes using 5-pinhole collimators and processed with 3D-OSEM algorithm using 4 subsets and 5 iterations. 3-dimensional CT data was processed with modified Feldkamp software. The processed 3D-images were fused and displayed with VIVID software package installed to the Triumph data management. Animals were under isoflurane anesthesia (1.5%) with O₂ flow (1.5 l/min) and kept warm during the imaging with a heated animal bed.

Organ Biodistribution of ¹¹¹In-PN(783)10.0:

150 μl of ¹¹¹In-labeled PN(783)10 (400 μCi, ˜2 nmole) were injected to tumor-bearing animals by tail vein (IV). 24 hours or 48 hours later, animals were sacrificed, and tumors, blood, liver, spleen, stomach, kidneys, small intestine, lung, heart, tail, fat, and muscle, were collected. Radioactivity was measured with Perkin Elmer, Wizard2 2480 gamma counter.

Confocal Imaging:

The mCherry-HT-29 tumor sample was collected at 48 hours post IV injection with PN(497)10.0, and then cryosectioned with thickness of 5 μm. The tumor section was fixed with 4% PFA, mounted with 90% glycerol/10% PBS (at pH 8.5 for best fluorescein fluorescence), and stained with DAPI. Confocal imaging was performed on a Zeiss LSM510 laser scanning confocal microscope (Zeiss Axiophot, Carl Zeiss, Jena, Germany). A 405 nm diode Laser, 488 nm argon laser, and 561 nm diode laser were used for the excitation of DAPI, fluorescein, and mCherry, respectively. A primary dichroic HFT 405/488/561 was used in combination with an LP420 emission filter for DAPI, BP505-530 for fluorescein, and LP575 for mCherry. Images were analyzed with ImageJ64.

Brain Vascular Phase Imaging (Angiography):

Craniotomies in C57Bl/6J wildtype mice (from Jackson Laboratory, Bar Harbor, Me., USA, 3-4 months old) were performed with minor modifications (see Skoch et al., “In vivo imaging of amyloid-beta deposits in mouse brain with multiphoton microscopy”, Methods in molecular biology (Clifton, N.J.) 299, 349-363, 2005). To summarize, animals were anesthetized using 2% isoflurane in balanced oxygen, and then a 5 mm diameter skull flap was removed. A craniotomy was performed, and the exposed brain area was covered by a 8 mm round glass coverslip, which was sealed to the skull with dental cement (see Spires-Jones et al., “Monitoring protein aggregation and toxicity in Alzheimer's disease mouse models using in vivo imaging”, Methods (San Diego, Calif.) 53, 201-207, 2011; and Fukumura, et al. “Tumor induction of VEGF promoter activity in stromal cells”, Cell 94, 715-725, 1998). This procedure allowed a transparent window into the mouse brain for use with in vivo microscopy of the cerebrovasculature. Mice were allowed 2-3 weeks for complete recovery after the craniotomy prior to imaging.

For imaging, mice were anesthetized with 2% isoflurane in balanced oxygen and secured in a custom stereotaxic frame, which fit into the microscope stage. The cerebrovasculature was imaged using the Olympus FluoView FV1000MPE multiphoton laser-scanning system mounted on an Olympus BX61WI microscope (Olympus, Tokyo, Japan). A DeepSee Mai Tai Ti:sapphire mode-locked laser (Mai Tai; Spectra-Physics, Fremont, Calif.) produced two-photon fluorescence with 800 nm excitation. The vessels were imaged at depth of 45 to 100 μm from the surface of the brain.

2 nmole of PN(497)/10.0 probe (300-400 μl) was injected retro-orbital into the anesthetized mouse. A time course was taken for up to 70 minutes post injection. Images were acquired using the Fluoview software and analyzed using ImageJ.

Imaging Tumor Interstium:

Dorsal skinfold chamber (DSFC) tumors were grown in female nude mice (nu/nu; 25-30 g; 6-8 weeks old) with modifications from previously published techniques (see Fukumura et al., “Tumor induction of VEGF promoter activity in stromal cells”, Cell 94, 715-725, 1998; and Marangoni et al., “The transcription factor NFAT exhibits signal memory during serial T cell interactions with antigen-presenting cells”, Immunity 38, 237-249, 2013). 10⁶ mCherry-HT-29 tumor cells in matrixgel (BD) were subcutaneously injected in the back of mice ˜1.5 cm left of the dorsal midline approximately halfway from the neck to the tail base. 4 days later, DSFCs were installed in a way that the tumors were centered in the imaging window of the chamber and accessible to longitudinal investigation by MP-IVM. On days 2, 3, and 4 days after tumor DSFC implantation, when tumors were typically 3 mm in diameter, image stacks of tumor tissue were recorded under general anesthesia with Ketamine and Xylazine. 100 μl (10 nmole) of PN(497)10.0 was injected (IV, tail vein).

Multiphoton excitation was obtained through DeepSee and MaiTai Ti:sapphire lasers (Newport/Spectra-Physics) tuned to 920 and 1000 nm to excite all fluorescent probes used. Stacks of 11 square optical sections with 4 μm z-spacing were acquired every 20 seconds on an Ultima IV multiphoton microscope (Prairie Technologies) using a 20×/0.95 NA lens with optical zoom of up to 1× to provide image volumes 30 μm in depth and 200 μm in width. Emitted fluorescence was detected through 460/50, 525/50, 595/50, 660/40 band-pass filters and non-descanned detectors to generate four-color images. Sequences of image stacks were transformed into volume-rendered, time-lapse movies with Imaris software (Bitplane).

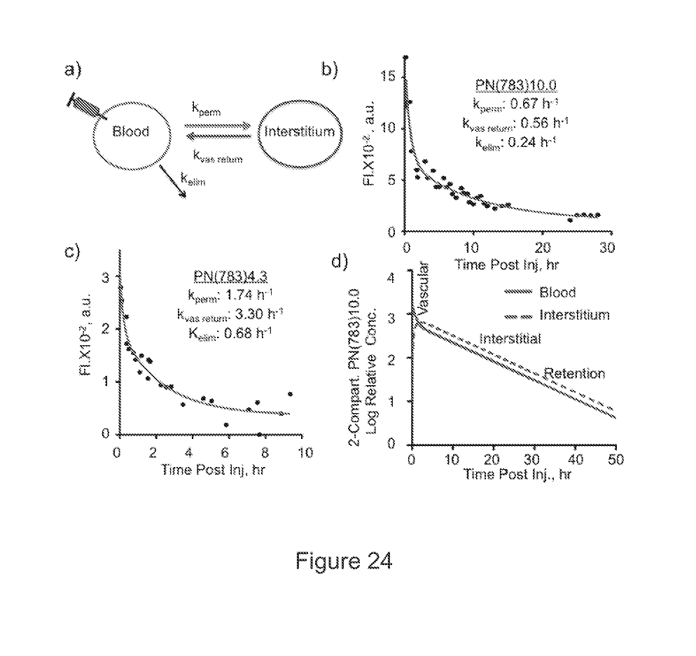

Two Compartment Model, See FIG. 24.

Serum fluorescence data from FIG. 24b and FIG. 24c was analyzed using the two-compartment model as described in Rosenbaum, S. E. Basic Pharmacokinetics and Pharmacodynamics: An Integrated Textbook and Computer Simulations, (John Wiley and Sons, Hoboken, N.J., 2011). Data were first fit to biexponential equation, yielding values of Alpha, Beta (apparent decay constants) and values of A and B as shown in equations 1 and 2 below.

$$General Biexponential Equation \text{:} Cp = A \cdot {e}^{-} \text{?} + B \cdot {e}^{-} \text{?}$$

$$Equation 1 , PN ( 783 ) 10 \text{:} {c}_{p} = 1039.5 exp ( - 1.361 t ) + 595.2 exp ( - 0.09903 t )$$

$$\text{?} \text{indicates text missing or illegible when filed}$$

PN(783)4.3: cₚ=112.8exp(−5.301t)+161exp(−0.425t) Equation 2,

By the relation of macro constants and micro constants,

$$k \text{?} - \frac{A β + B α}{A + B} , k \text{?} - \frac{αβ}{k \text{?}} , k \text{?} - α + β - k \text{?} - k \text{?} , \\ \text{?} \text{indicates text missing or illegible when filed}$$

micro constants k's can be obtained, and the half-life was calculated by

$$A 1 \text{/} 2 = 0.693 \text{/} k ,$$

see above

TABLE S1
Summary of constants for PN(783)4.3 and PN(783)10.0
obtained with The two-compartment model

| | PN(783)4.3 | | PN(783)4.3 | | PN(783)10.0 | | PN(783)10.0 | |
|---|---|---|---|---|---|---|---|---|
| Constant | rate | | half-life | | rate | | half-life | |
| Alpha | 5.30 | h⁻¹ | 0.13 | h | 1.36 | h⁻¹ | 0.51 | h |
| Beta | 0.42 | h⁻¹ | 1.64 | | 0.099 | h⁻¹ | 7.0 | h |
| ¹¹¹In- | | | | | | | 7.8 | h |
| PN(783)10 | | | | | | | | |
| elimination* | | | | | | | | |
| k perm | 1.74 | h⁻¹ | 0.40 | h | 0.67 | h⁻¹ | 1.02 | h |
| k vas return | 3.30 | h⁻¹ | 0.20 | h | 0.56 | h⁻¹ | 1.23 | h |
| k elim | 0.68 | h⁻¹ | 1.01 | h | 0.24 | h⁻¹ | 4.2 | h |

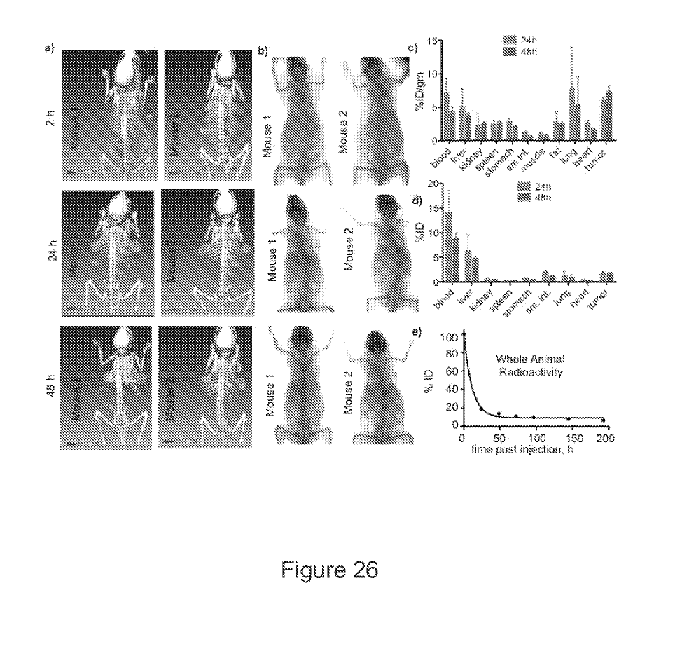

*See FIG. 26e.

### Overview of Example 6

PEG-like Nanoprobes (PN's) are pharmacokinetically and optically tunable materials whose disposition in biological systems can be determined by fluorescent or radioactive imaging modalities. PN's are synthesized by attaching different fluorochromes and PEG polymers of different molecular weights to a (DOTA)Lys-Cys dipeptide scaffold, yielding PN's with different sizes, pharmacokinetics, and excitation and emission maxima. PN's exploit the PEG-fluorochrome shielding effect, where PEG polymers are used to block the interactions of fluorochromes with each other or biomolecules. PN's were used to image brain capillaries (2-photon microscopy), tumor capillary permeability (intravital microscopy), and the tumor EPR effect (¹¹¹In-PN) by SPECT imaging. DOTA provides a radiolabeling option that not only allows SPECT imaging, but allows ready determination of PN biodistribution and elimination. ¹¹¹In-PN with a diameter of 10 nanometers exhibited a combination of a long circulation time and low whole body retention, with a low hepatic uptake (despite being nearly double the 5.4 nm of albumin), and virtually no kidney retention (despite employing dipeptide scaffold). PN's provide a unique combination of pharmacokinetic tunability (through PEG selection), spectral tunablity (through fluorochrome selection) and easy radiolabeling (DOTA chelation). PN's offer a simple and superior chemistry for obtaining passively targeted, pharmacokinetically tunable fluorochromes and/or radiometals.

In Example 6, we introduce passively targeted, fluorescent and/or radioactive nanomaterials with PEG-determined sizes in the nanometer range and termed “PEG-like Nanoprobes” (PN's). PN's are synthesized by attaching different fluorochromes and different PEG polymers to a (DOTA)Lys-Cys dipeptide scaffold, yielding PN's with different sizes, pharmacokinetics, and excitation and emission maxima. PN's are based on the discovery that PEG's (MW>5 kDa), when covalently linked to fluorochromes, block the interactions of fluorochromes with each other and blocking their interactions with biomolecules and cells. (See Guo et al. (2012) “PEG-Fluorochrome Shielding Approach for Targeted Probe Design,” JAGS). PN's achieve spectral flexibility by endowing different fluorochromes with PEG-like rather than fluorochrome-like behavior in vitro and in vivo. In Example 6, we show how PN's can employ a modular design approach, with a fixed scaffold adorned by a variable fluorochrome and a variable PEG, an approach which yields pharmacokinetic and spectral flexibility, a large number of potential uses (fluorescent and radioactive imaging), and a high potential for clinical safety.

Although a wide range of approaches has been explored for obtaining passive, non-receptor mediated fluorochromes or radiometals, all have important limitations. Novel nanomaterials (e.g. nanoshells, carbon nanotubes, dendrimers, quantum dots) suffer from a lack of knowledge about their toxicity and/or elimination and a lack of clinical history. Fluorescent dextrans have been widely used. However, dextrans induce histamine release in rodents, altering capillary permeability. Clinical use of dextrans is complicated by anti-dextran antibodies and dextran induced anaphylaxis. In contrast, PEG polymers employed by PN's have little if any immunogenicity and are widely recognized as safe due to their extensive use in parenteral pharmaceuticals.

As a carrier for the passive delivery of diagnostic agents albumin is not ideal because of an albumin receptor, and because modified albumins can be recognized as abnormal versions of normal albumin and cleared by scavenger receptors. Reversible complexation with albumin provides another general technique for obtaining passively targeted, long-circulating diagnostic agents. Albumin complexes with (ICG) or dyes (Evans Blue) before or after injection. However, the reversibility means transcapillary passage and interstitial accumulation can be due to the slow transport of the major albumin-bound form or a fast passage of the minor, low molecular weight species. Albumin-based approaches, whether covalent or reversible complexation, cannot be used to understand the size dependence of biological processes preclinically, or permit optimization of size and pharmacokinetics for clinical uses.

### Example 6

### Results

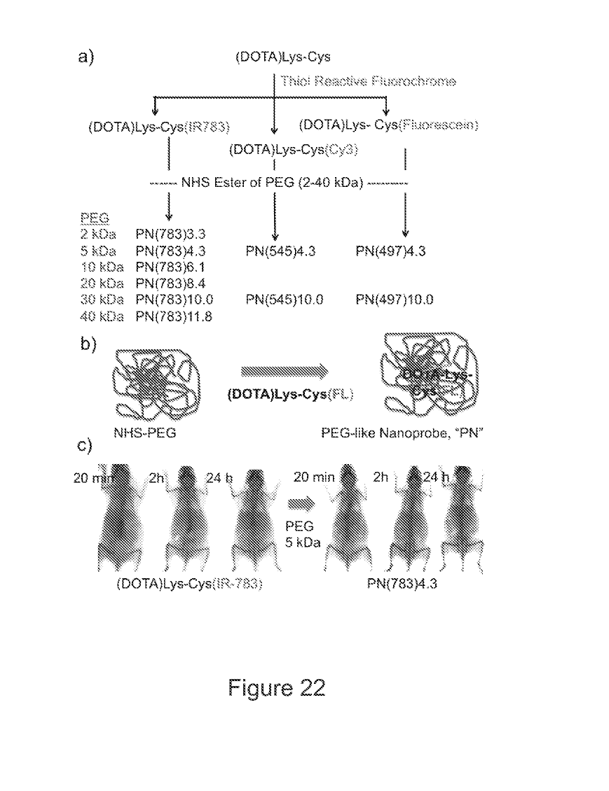

PEG-like Nanoprobes (PN's) employ a modular synthetic strategy (see FIG. 22a) where a variable, fluorochrome is reacted with the cysteine thiol of a (DOTA)Lys-Cys peptide (see FIGS. 15-18). (All peptides are C-terminal amides with the final —NH₂ omitted.) Peptides are denoted (DOTA)Lys-Cys(FL), where FL is a fluorochrome: IR-783, Cy3 or fluorescein. (DOTA)Lys-Cys(FL) peptides are then reacted with NHS esters of a PEG polymer of different molecular weights (see FIGS. 19-21). PN's and their properties are summarized in FIG. 22a and Table 5. DOTA provides a radiolabeling option the value of which is explained below.

Two nomenclatures are employed, a PN nomenclature and a peptide nomenclature. PN(783)4.3, (column 1 of Table 5) indicates a PEG-like Nanoprobe with an absorption maxima of 783 nm and hydrodynamic diameter of 4.3 nm. With peptide nomenclature PN(783)4.3 is (DOTA)Lys(PEG 5 kDa)-Cys(IR783), see column 2 of Table 5.

Key features of PN's are summarized in FIG. 22b. Since PEG's are extended, water infiltrated structures with solution diameters are far larger than their molecular weights, the PEG polymer confers nanometer sizes upon PN's. For example, reaction of the (DOTA)Lys-Cys(IR-783) peptide (diameter=1.1 nm), with a 5 kDa PEG yielded PN(783)4.3 (diameter=4.3 nm). Hence the PEG-based expansion of volume of was 59 fold, (4.3/1.1).

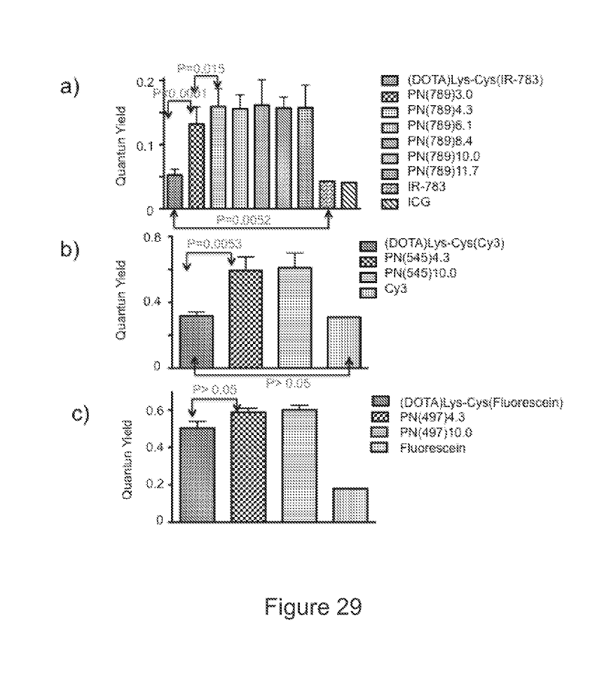

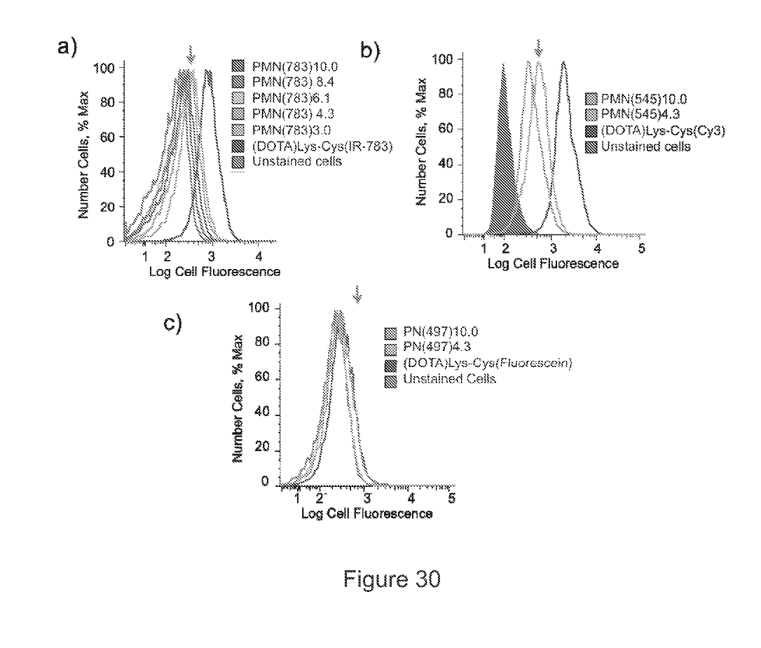

Attachment of PEG increased the quantum yields of fluorochromes as shown in FIG. 29 and summarized in Table 5. The attachment of a 2 kDa PEG to (DOTA)Lys-Cys(IR-783) (i.e. yielding PN(783)3.0), resulted in significantly less improvement in quantum yield than larger PEG's (see FIG. 29); this was scored as incomplete PEG fluorochrome shielding and only PN's made with larger PEG's (PEG=5 kDa or greater) were studied further. Previous studies have shown than attachment of a 5 kDa PEG's results a loss absorption spectra PBS that are similar to unstacked spectra obtained in methanol, suggesting quantum yield improvement reflects a blockage of fluorochrome/fluorochrome stacking (see Guo et al., “The PEG-Fluorochrome Shielding Approach for Targeted Probe Design,” J Am Chem Soc 2012, 134(47): 19338-19341). PEG decreased the non-specific binding to HT29 cells, scored as the percent of cells above the cutoff for unstained cells seen with FACS (Table 5, FIG. 30). PEG reduction of binding was not detectable when the (DOTA)Lys-Cys(Fluorescein) peptide was PEGylated, reflecting a lack of nonspecific binding with this peptide and/or a higher intrinsic cell fluorescence at lower wavelengths.

A dramatic illustration of the effect of attaching a 5 kDa PEG to the (DOTA)Lys-Cys(IR-783) peptide is its ability enhance elimination following an IV injection. As shown in FIG. 22c, the resulting PN, PN(783)4.3 PEGylation with a 5 kDa PEG also enhanced the elimination of IM administered peptides. “PEG-like Nanoprobes” employ PEG to decrease fluorochrome/fluorochrome interactions nonspecific interactions with cells and to enhance the elimination of fluorochrome bearing peptides.

To demonstrate the ability of PEG to tune (vary) PN size, the diameters of PN's synthesized using PEG's of different molecular weights (FIG. 22a) and IR-783 were determined by FPLC gel-filtration chromatography (FIG. 23a). The column was calibrated with globular protein standards, and globular protein equivalent molecular weights (in kDa) were obtained (Table 5, column 5). These were converted to PN diameters in nm using the relationship: Radius in nm=0.066M¹¹³ where M is the molecular weight of a globular protein expressed in daltons (see Erickson, “Size and Shape of Protein Molecules at the Nanometer Level Determined by Sedimentation, Gel Filtration, and Electron Microscopy”, Biological Procedures Online 2009, 11(1): 32-50). PN's diameters ranged from 3.0 to 11.8 nm (see FIG. 23a). Using PEG's of 5 kDa and 30 kDa, selected for their low polydispersity, PN's were synthesized using IR-783, Cy3 and Fluorescein (see FIG. 22a) and their sizes determined (see FIGS. 23a, 23b, 23c). With the 5 kDa PEG and these three fluorochromes (see the magenta chromatograms in FIGS. 22a, 23b and 23c) PN diameters were 4.3 nm. With the 30 kDa PEG and these three fluorochromes (blue chromatograms, 23a, 23b, 23c), PN diameters were now 10.0 nm. For reference, the diameter of a 67 kDa albumin determined by this method was 5.4 nm.

To see if the tunable size of PN's could be translated into tunable pharmacokinetics, it was essential to first establish that PN's circulated at their variable, PEG-determined pre-injection sizes. FPLC chromatograms of PN(783)10.0, PN(783)6.1 and PN(783)4.3, at their pre-injection sizes and at varying times post injection, are shown in FIGS. 23d, 23e and 23f. By using PN's absorbing at 783 nm, chromatograms before and after injection (i.e. serum of injected mice) can be compared, since there are no compounds in serum that absorb at this wavelength. With the FPLC chromatograms of PN(783)10.0 (2d), peaks from a pre-injection sample or serum at 21 or 40 minutes were at identical elution volumes, indicating that this PN circulates at its pre-injection size. Similarly, peaks for PN(783)6.1 (see FIG. 23e) and PN(783)4.3 (see FIG. 23f) where identical for pre-injection samples and for sera from injected mice. Thus molecular weight of the PEG chosen determines PN dimensions and those dimensions are maintained after injection.

Since PN's circulate at variable PEG-determined sizes, they undergo transcapillary passage as their injected form as shown in FIG. 23g. In contrast, albumin-binding compounds used in the determination transcapillary passage exist as albumin bound and free forms. Examples include fluorophores (ICG), chromophores (Evans Blue), and Gd chelates (gadofosveset). PN's are size variable, multimodal nanomaterials for the determination capillary permeability without the uncertainties by presented by albumin bound and free forms in circulation.

To assess the relationship between PN dimensions and transcapillary passage, the classic two-compartment pharmacokinetic model (see Rosenbaum, Basic Pharmacokinetics and Pharmacodynamics: An Integrated Textbook and Computer Simulations, (John Wiley and Sons, Hoboken, N.J., 2011) (see FIG. 24a) was applied to PN(783)4.3 and PN(783)10.0, see FIGS. 24b and 24c. After injection, blood concentrations exhibit an initial fast exponential decay (vascular escape), followed by a slow exponential decay (whole body clearance). The three microscopic rate constants for the two-compartment model are given in FIGS. 24b and 24c. Further details on the two-compartment model are provided above with summary of all pharmacokinetic constants (see Table 51). The rapid initial fall of PN blood concentration is due to vascular escape, as occurs with fluorescent dextrans of a similar size. Although the circulation times of PN's in mice appear modest, our values are consistent with studies using fluorescent dextrans in mice. Since a 40 kDa dextran has plasma half-life of 10 hours, considerably longer half-lives of PN's are expected if used clinically.

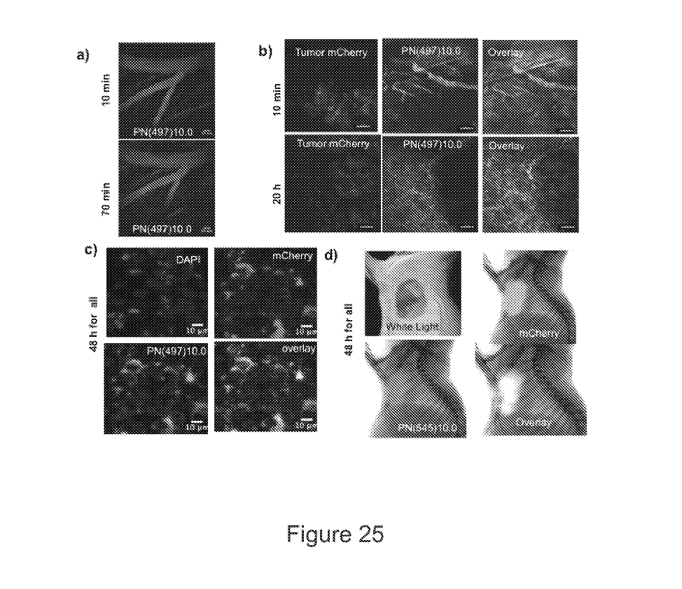

The concentrations of PN(783)10.0 in the blood and interstitial compartments using the values from FIG. 24b are shown in FIG. 24d. Three pharmacokinetic phases shown are a vascular phase (approximately for 1 h post injection), an interstitial phase at (at 10-25 h), and an enhanced permeability retention (EPR) based uptake by a tumor at 48 hours. These phases were examined with SPECT imaging and radioactive biodistribution studies in FIG. 25 and with fluorescence imaging techniques in FIG. 26.

To demonstrate a multimodal imaging capability, the ability of ¹¹¹In-PN(783)10.0 (or PN(783)10.0) to image the EPR effect of an HT29 tumor was determined by SPECT/CT (see FIG. 25a) and surface fluorescence (see FIG. 25b). Initially (2 hours post injection) by SPECT or surface fluorescence, PN783)10.0 was broadly distributed, consistent with PN(783)10's vascular and interstitial distribution seen with the two-compartment model (see FIGS. 24b, 24d). At 48 hours, PN(783)10.0 was retained in the tumor with SPECT and surface fluorescence, reflecting the high tumor concentrations (7.28±0.93% ID/gm) and the proximity of the tumor to the surface (see FIG. 25c).

Biodistribution studies with ¹¹¹In-PN(783)10.0 are shown in FIGS. 25c and 25d. At 48 hours post injection, 4.71±0.38% of dose was in the liver (see FIG. 25d), despite the fact that with a diameter of 10 nm this PN is considerably larger than albumin (diameter=5.4 nm). In addition only 0.44±0.02% of the injected dose was in the kidney, which typically accumulates high levels of radiolabeled peptides due renal peptide transporters. Whole body radioactivity decreased with a half-life of 7.8 hours and was 13.75±0.74% of injected dose by 48 hours (see FIG. 25e).

To image the vascular phase, PN(497)10.0 was used for two-photon intravital microscopy of brain capillaries (see FIG. 26a), since the blood brain barrier blocks interstitial accumulation. Vessel fluorescence decreased as imaging time increased from 10 to 70 minutes, reflecting a decrease in the blood concentration of PN(497)10.0 from transcapillary passage (see FIG. 24b). To further examine the vascular and interstitial phases, a dorsal skinfold chamber was used for the intravital, two photon microscopy of an mCherry expressing HT29 tumor (FIG. 25b). At ten minutes post injection, PN(497)10.0 (green) was confined to the vasculature at the periphery of the mCherry tumor(red). At 20 hours post injection, the PN was seen in the interstitium at the tumor periphery.

To determine the cells responsible for the EPR accumulation of PN(783)10.0 seen with SPECT and surface fluorescence (see FIG. 25), we employed confocal microscopy of mCherry/HT29 tumor sections (see FIG. 26c). PN(545)10.0 was seen in mCherry/HT29 cells, presumably by fluid phase pinocytosis, since PEG does not bind known receptors. The retention of PN(545)10.0 was also evident from surface fluorescence measurements as shown in FIG. 26d. With skin removed, an overlay of tumor mCherry (green) and PN(545)10.0 (purple) gave a white superimposition.

### Example 6

### Discussion

By using a single PEG polymer of sufficient length (5 kDa or greater), PN's achieve the properties of PEG in vitro and in vivo that enable them to be described as “PEG-like Nanoprobes.” In vitro, PEG-like properties include an increased quantum yield, a decreased binding to cultured cells (see Table 5), and the attainment of sizes in the nanometer size range. In vivo, PEG-like properties include extended circulation times, low hepatic uptake and excellent whole body elimination.

PN's provide a unique combination of pharmacokinetic tunability and low whole body retention. ¹¹¹In-PN(783)10.0 had only 4.71±0.38% of the injected dose in the liver (48 hours post), in spite of the fact that its diameter is nearly twice that of albumin (5.4 nm). ¹¹¹In-PN(783)10.0 is therefore unlike high molecular weight dextrans, which have extended blood half-lives but undergo eventual hepatic uptake, principally by Kupffer cells. ¹¹¹In-PN(783)10.0 is unlike the low molecular weight near infrared fluorochrome ICG, which undergoes rapid hepatic clearance as the albumin bound complex. Finally, ¹¹¹In-PN(783)10.0 is unlike many radiolabeled peptides that are excellent substrates for renal peptide transporters, and which make the kidney the organ of highest tracer concentration and organ of dose limiting toxicity. In contrast, ¹¹¹In-PN(783)10.0 exhibited a renal retention of only 0.44±0.02% (48 hours post). Based on their low renal and hepatic accumulation, the model for PN's where PEG shields both the Lys-Cys peptide and the attached fluorochrome (see FIG. 22b) is supported.

At least three potential applications of PN's can be considered. First, the blood half-life control provided by fluorochromes PN's could enable a long duration fluorescent angiography in neurosurgery or reconstructive surgery. ICG, with a blood half-life of 4 minutes, is now used. Here PN's were used for fluorescent angiography in normal brain and the HT-29 tumor (see FIGS. 25a, 25b). Second, PN's might be used to image tumor the EPR effect (see FIG. 26a), resolving the issue of its existence and magnitude with human tumors. Long-circulating nanomedicine therapeutics, liposomes and polymer conjugates, approved or in clinical trials, may utilize the EPR effect in part for their efficacy.

However, a broad and important class of uses for PN's lies in the determination capillary permeability and endothelial function that can be aberrant in relatively common conditions including diabetes, sepsis and ischemic insult. PN's are ideal for the determination of capillary permeability (by SPECT or fluorescence) because they exist post injection as PEG-determined, size variable, pharmacokinetically tunable materials (see FIG. 23). Here the radiolabeling option may lead to a unique path to clinical development of PN's as fluorescent or radioactive capillary permeability agents, permitting microdose pharmacokinetic studies as a function of PN size (see FIG. 24). This would enable selection of a PN with optimal pharmacokinetics for fluorescent capillary permeability imaging. Thus PN's exhibit pharmacokinetic tunability, spectral tunability and a radiolabeling option, a combination that has not been achieved with previous nanomaterials used for passive pharmacokinetic targeting. This unique combination of properties and capabilities may lead to their use in various areas of clinical practice.

TABLE 5
SUMMARY OF PROPERTIES OF PEG-LIKE NANOPROBES

| PEG-like | | | | | | | |
|---|---|---|---|---|---|---|---|
| Nanoprobe | | | MW, | Volume | | Quant. | Cell |
| “PN” | Peptide | Abs/em | (kDa) | FPLC, | | Yield | NSB |
| Nomenclature | Nomenclature | (nm) | Observed | (FIG. 23a) | | (FIG. 29) | (FIG. 30) |
| Not | (DOTA)Lys- | Not | 0.6356 | Not | | Not | Not |
| Applicable | Cys | Applicable | | Applicable | | Applicable | Applicable |
| Not | (DOTA)Lys- | 789/807 | 1.3258 | 0.600 | kDa | 0.053 | 95% |
| Applicable | Cys(IR-783) | | | 1.10 | nm | | |
| PN(783)3.0 | (DOTA)Lys(PEG2K)- | 789/810 | 3.34 | 11.2 | kDa | 0.13 | 50% |
| | Cys(IR-783) | | | 3.0 | nm | | |
| PN(783)4.3 | (DOTA)Lys(PEG5K)- | 789/810 | 6.29 | 35.2 | kDa | 0.16 | 20% |
| | Cys(IR-783) | | | 4.3 | nm | | |
| PN(783)6.1 | (DOTA)Lys(PEG10K)- | 789/810 | 11.41 | 100.7 | kDa | 0.16 | 14% |
| | Cys(IR-783) | | | 6.1 | nm | | |
| PN(783)8.4 | (DOTA)Lys(PEG20K)- | 789/810 | 21.28 | 262.5 | kDa | 0.16 | 14% |
| | Cys(IR-783) | | | 8.4 | nm | | |
| PN(783)10.0 | (DOTA)Lys(PEG30K)- | 789/810 | 30.62 | 435.4 | kDa | 0.16 | 18% |
| | Cys(IR-783) | | | 10.0 | nm | | |
| PN(783)11.7 | (DOTA)Lys(PEG40K)- | 789/809 | 45.00 | 690 | kDa | 0.16 | |
| | Cys(IR-783) | | | 11.7 | nm | | |
| Not | (DOTA)Lys- | 544/559 | 1.2138 | 0.6 | kDa | 0.32 | 98% |
| Applicable | Cys(Cy3) | | | | | | |
| PN(545)4.3 | (DOTA)Lys(PEG5K)- | 545/560 | 6.18 | 35 | kDa | 0.59 | 31% |
| | Cys(Cy3) | | | | | | |
| PN(545)10.0 | (DOTA)Lys(PEG30K)- | 545/560 | 30.51 | 435 | kDa | 0.61 | 15% |
| | Cys(Cy3) | | | | | | |
| Not | (DOTA)Lys- | 497/521 | 1.0626 | 0.6 | kDa | 0.50 | 0.4% |
| Applicable | Cys(FI) | | | | | | |
| PN(497)4.3 | (DOTA)Lys(PEG5K)- | 497/521 | 6.04 | 35 | kDa | 0.59 | 0.2% |
| | Cys(FI) | | | | | | |
| PN(497)10.0 | (DOTA)Lys(PEG30K)- | 497/521 | 30.36 | 435 | kDa | 0.60 | 0.1% |
| | Cys(FI) | | | | | | |

### Example 7

### Synthesis of PEG-Like Nanoprobes

In this Example, we prepared multimodal, pharmacokinetically and optically tunable nanomaterials using desferoxamine (DFO) and ⁸⁹Zr⁴⁺.

In Example 6 above, we show how PEGylated fluorochromes can be made with different PEG's and different fluorochromes. In Example 6, we demonstrated the synthesis of PEGylated fluorochromes of the general formula (DOTA)Lys(PEG)-Cys(FL), where “FL” is a fluorochrome like IR-783 or Cy3 or Fluorescein and PEG is polymer PEG chain with molecular weight between about 2 kDa and 40 kDa. By varying the PEG molecular weight, pharmacokinetic variation and tunability is obtained. By varying the fluorochrome (FL), optical properties are varied and spectral tunability is obtained.

Unfortunately, there are no long-lived, positron emitting metal ions for which DOTA has a high affinity. DOTA has a high affinity for ¹¹¹In³⁺ (half-life=2.7 days, good for SPECT imaging) and ⁶⁸Ga³⁺ (half-life=68 minutes, good for PET imaging).

To remedy the inability of DOTA to chelate long-lived, positron emitting isotopes, we have now replaced the DOTA with desferoxamine (DFO), to obtain materials with the general formula (DFO)Lys(PEG)-Cys(FL). The conjugation of desferoxamine (DFO) to proteins (e.g. antibodies and albumin), followed by chelation of the positron emitting ⁸⁹Zr⁴⁺ (half-life 78 hours), and imaging protein disposition by PET is now a widely accepted approach to imaging long circulating proteins. We have shown that these yield (⁸⁹Zr⁴⁺:DFO)Lys-Cys(FL) where FL=a fluorochrome, which can be used for PET imaging.

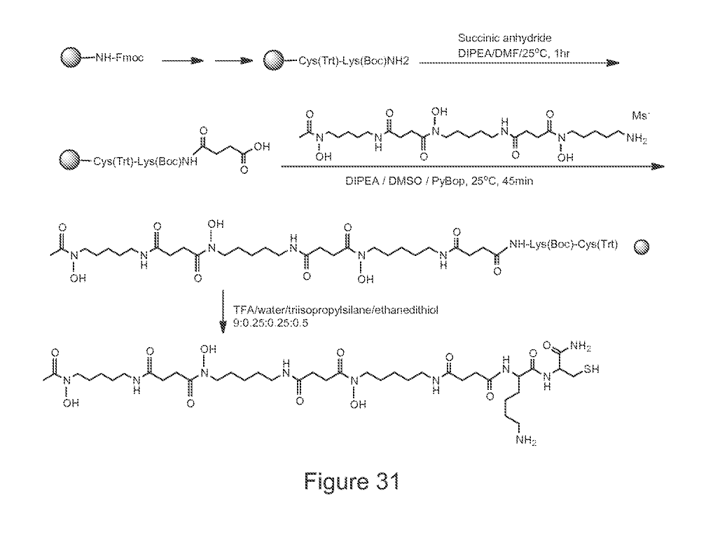

Synthesis of the DFO-Lys(NH2)-Cys(SH) Peptide (See FIG. 31)

The DFO-Lys(Boc)-Cys(Trt) peptide was manually synthesized on Rink Amide MBHA resin (0.15 mmol) with an Fmoc/t-Bu strategy using a polypropylene 5 mL disposable syringe fitted with a sintered frit. Coupling reactions employed 2 equiv. (relative to resin) of Fmoc-protected amino acid activated in situ with 2 equiv. of PyBOP and 4 equiv. of DiPEA in DMF (10 mL/g resin) for 1-2 hours. Coupling efficiency was assessed with picrylsulfonic acid. Fmoc groups were removed with a piperidine/DMF solution (1:4) for 4×10 min (10 mL/g resin). The N-terminal of the peptide was succinilated by succinic anhydride (8 eq) with the presence of DIPEA (8 eq) in DMF, while a carboxylic acid was generated for the attachment of DFO. PyBop (4 eq) and DIPEA (16 eq) in DMSO (1 ml) was pulled into the syringe and stayed in room temperature for 20 minutes. Then the solution of DFO-mesylate salt (4 eq) in DMSO (3 ml) was mixed with the PyBOP solution in the syringe and incubated under room temperature for overnight. DFO-Lys-Cys was released from the solid support with TFA/H2O/TIS/EDT 88:2:5:5 (2 h, 20 mL/g resin). The residue was precipitated and triturated with cold diethyl ether. A white solid could be obtained by centrifuge. The solid was purified further by HPLC with buffer B from 15% to 65% in 10 minutes, back to 15% B in 2 minutes, and isocratic for 3 minutes with a flow of 12 ml/min at λmax=226 nm on a column of Agilent, PLRP-S 100A, 15-20 μm, P/N: PL1812-6200. A double charged peak with MS 446.5 was found. Overall yield: 37.5%.

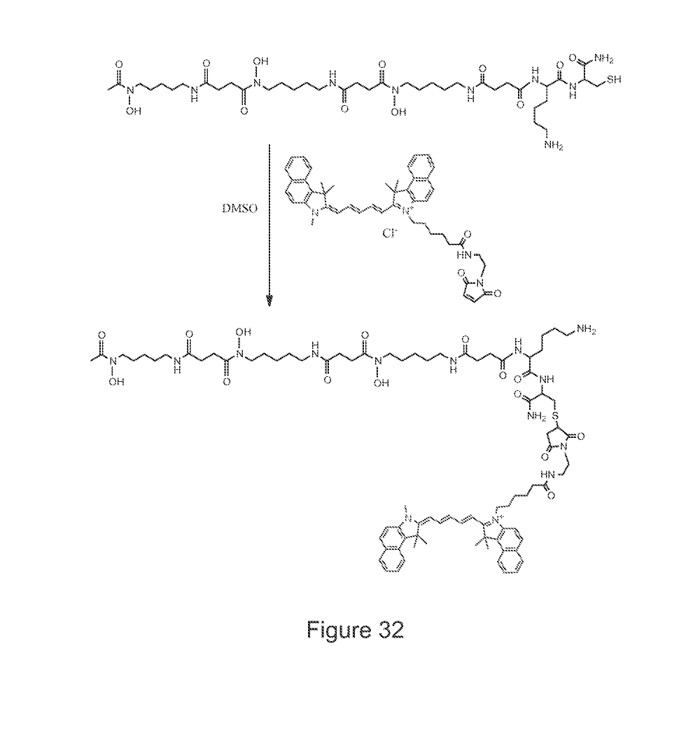

### Synthesis of DFO-Lys(NH2)-Cys(S-Mal-Cy5.5, Lumiprobe) (see FIG. 32)

DFO-Lys(NH2)-Cys(SH)-NH2 (5.7 mg, 6.4 umol) and Cy5.5-Maleimide (4.73 mg, 6.4 umol) was mixed in DMSO (0.7 ml) in the presence of DIPEA (7 ul, 40.3 umol) for overnight in room temperature under N₂. The product was purified by HPLC separation (a gradient of 20-100% buffer B in 10 minutes, back to 20% in 5 minutes and isocratic for 5 minutes, flow: 21 ml/min, 275 nm; column: Agilent, PLRP-S 100A, 15-20 μm, P/N: PL1812-6200). A blue powder was obtained after lyophilization with a yield: >90%; MS: C₈₄H₁₁₉N₁₄O₁₅S⁺, calculated: 1595.87. found: 798.7 [M+1]²⁺, 533 [M+2]³⁺.

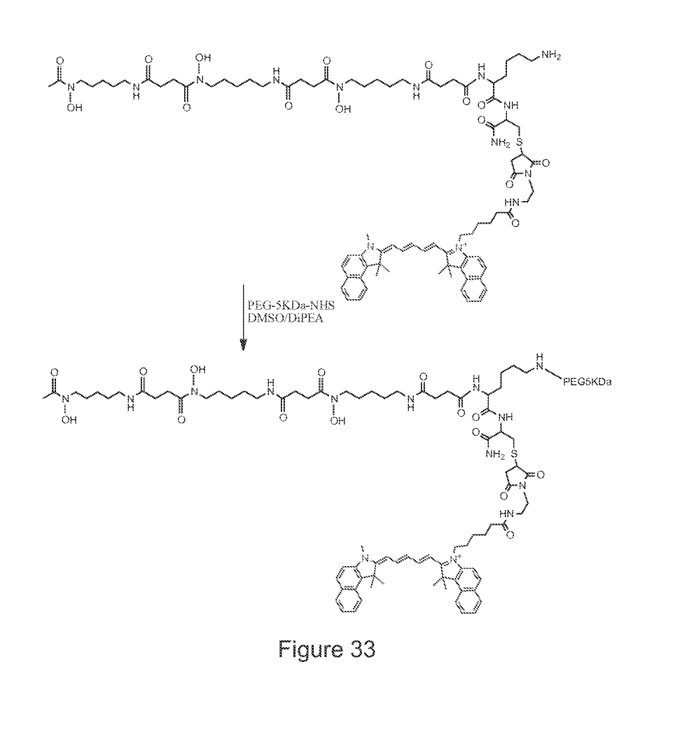

### Synthesis of DFO-Lys(PEG5KDa)-Cys(S-Mal-Cy5.5)-NH2 (see FIG. 33)

To a solution of DFO-Lys(NH2)-Cys(S-Mal-Cy5.5)-NH2 (1.28 mg, 0.8 umol in DMSO), was added the solution of m-PEG-5K-NHS (12 mg, 2.4 μmol, 3 eq). After DiPEA (7.37 μL, 42.4 umol, 53 eq to MSAP) was added, the reaction mixture was incubated for 3 days at room temperature. The product was purified by HPLC (gradients: 20-100% B in 10 minutes, back to 20% buffer B in 5 minutes, then isocratic for 5 minutes; flow, 21 ml/min, 675 nm, column: Agilent, PLRP-S 100A, 15-20 μm, P/N: PL1812-6200). Yield: >90%, MS: 6689.73 (multiple dispersed).

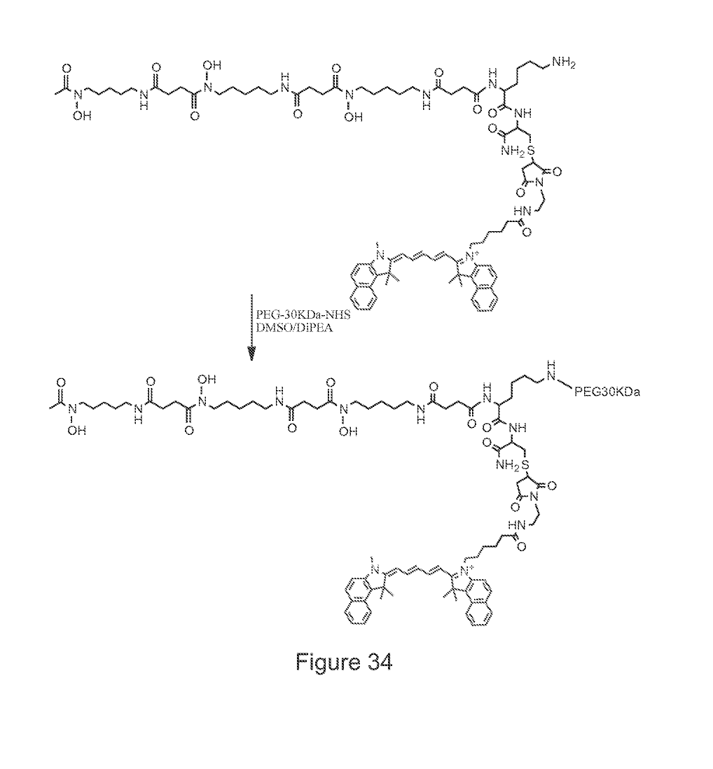

### Synthesis of DFO-Lys(PEG30KDa)-Cys(S-Mal-Cy5.5)-NH2 (see FIG. 34)

To a solution of DFO-Lys(NH2)-Cys(S-Mal-Cy5.5)-NH2 (0.8 umol, 1.28 mg, in DMSO), was added the solution of m-PEG-30K-NHS (72 mg, 2.4 μmol, 3 eq in DMSO 1 ml) and incubated for 3 days at room temperature in the presence of DiPEA (7.37 μL, 42.4 umol, 53 eq to MSAP). The product was purified by HPLC (20-100% buffer B in 10 minutes, back to 20% B in 5 minutes, then isocratic for 5 minutes; flow, 21 ml/min, 675 nm, column: Agilent, PLRP-S 100A, 15-20 μm, P/N: PL1812-6200). Yield: >90%, MS: 30,000 (multiple dispersed).

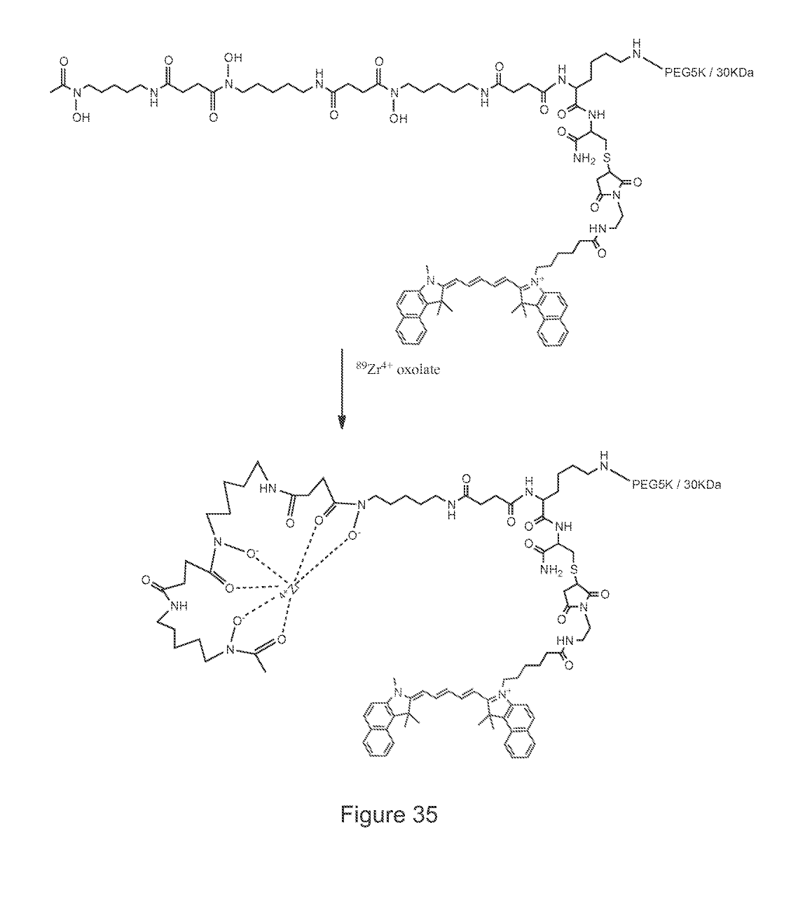

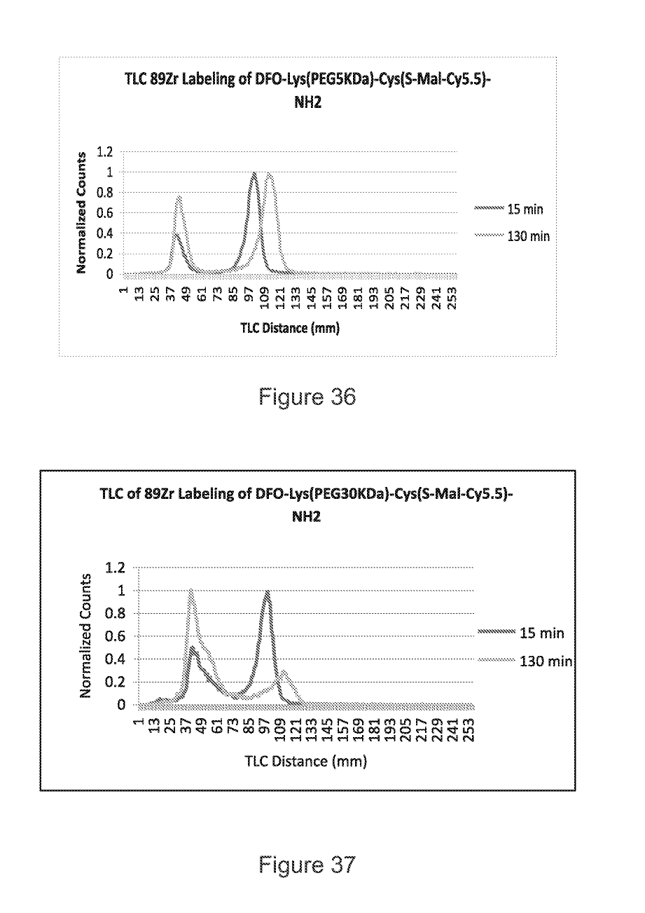

### ⁸⁹Zr Labeling of Compounds DFO-Lys(PEG5KDa)-Cys(S-Mal-Cy5.5)-NH2 and DFO-Lys(PEG30KDa)-Cys(S-Mal-Cy5.5)-NH2 (see FIGS. 35-37)

A solution of ⁸⁹Zr-oxalate (250 μl, 287 μCi) was neutralized by Na₂CO₃ (1 M, in chelexed water, 170 μl) until the up to pH 8.5. Two aliquots were made for the labeling of 2 nmol DFO-Lys(PEG5KDa)-Cys(S-Mal-Cy5.5)-NH2 and DFO-Lys(PEG30KDa)-Cys(S-Mal-Cy5.5)-NH2 by adding their stock solutions in chelexed water respectively. They were incubated under room temperature for 2 hours with radioactive TLC monitoring. The labeling yield were 40% for DFO-Lys(PEG5KDa)-Cys(S-Mal-Cy5.5)-NH2 and 75-80% for DFO-Lys(PEG30KDa)-Cys(S-Mal-Cy5.5)-NH2. The labeled compounds were purified by PD-10 column with fraction collection.

Although the present invention has been described in detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1-39. (canceled)

40. A fluorescent compound having a formula selected from the group consisting of:
(a) formula (I): 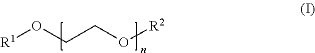
wherein R¹ is a fluorescent moiety having an absorption wavelength maxima in the range of 450 to 1500 nanometers, and
wherein R² is a non-reactive moiety, and
wherein n is an integer;
(b) formula (II): 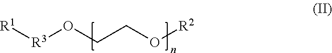
wherein R¹ is a fluorescent moiety having an absorption wavelength maxima in the range of 450 to 1500 nanometers, and
wherein R² is a non-reactive moiety, and
wherein R³ is a scaffold including an amino acid group, and
wherein n is an integer; and.
(c) formula (III): 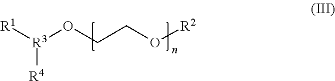
wherein R¹ is a fluorescent moiety having an absorption wavelength maxima in the range of 450 to 1500 nanometers, and
wherein R² is a non-reactive moiety, and
wherein R³ is a scaffold including an amino acid group, and
wherein R⁴ is selected from chelates, proteins, enzymes, peptides, antibodies, and drugs that can target a site in a subject, and
wherein n is an integer.

41. The compound of claim 1, wherein n is selected such that chain (C) in the compound 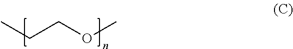
has a molecular weight of 2,000 daltons or more.

42. The compound of claim 1, wherein n is selected such that after intravenous administration of the compound to a mammal, the compound undergoes renal elimination or clearance is by macrophages of the reticuloendothelial system of the mammal.

43. The compound of any of claim 1, wherein chain (C) in the compound 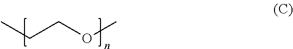
shields R¹ from reaction with biological molecules.

44. The compound of claim 1, wherein the fluorescent moiety has an absorption wavelength maxima in the range of 550 to 850 nanometers.

45. The compound of claim 1, wherein the compound has a quantum yield of greater than 0.1.

46. The compound of claim 1, wherein the compound has a molecular volume that correlates with an apparent molecular weight greater than about 10,000 daltons when analyzed by fast protein liquid chromatography and globular protein standards.

47. The compound of claim 1, wherein R² is selected from the group consisting of C₁-C₂₀ alkyl and aryl.

48. The compound of claim 1, wherein the fluorescent moiety is selected from the group consisting of a cyanine dye, a carbocyanine dye, a CyAL dye, and fluorescein.

49. The compound of claim 1, wherein R⁴ is a chelate.

50. The compound of claim 1, wherein the compound has a hydrodynamic diameter in the range of 1 to 100 nanometers.

51. The compound of claim 1, wherein the scaffold is a peptide including two or more residues selected from alanine, arginine, aspartate, cysteine, glycine, and lysine.

52. A method for imaging a region of interest of a subject, the method comprising:
administering to the subject a compound of claim 1, wherein the compound enters the region of interest of the subject;
directing light into the subject;
detecting fluorescent light emitted from the subject; and
processing the detected light to provide an image that corresponds to the region of interest of the subject.

53. The method of claim 13, wherein the light directed into the subject has a wavelength in the range of 450 to 1500 nanometers.

54. The method of claim 13, wherein the fluorescent light is emitted via two-photon-excited fluorescence.

55. The method of claim 13, further comprising imaging the subject with a second imaging method selected from positron emission tomography, single-photon emission computed tomography, magnetic resonance imaging, computerized tomography, optical imaging, and ultrasound.

56. The method of claim 13, wherein the region of interest of the subject includes a tumor.

57. The method of claim 17, wherein if the compound binds to the tumor, the method further comprises administering to the subject a therapeutically effective amount of a cytotoxic material comprising a compound of claim 1 associated with a cytotoxic agent.

58. A method for treatment of a tumor in a subject, the method comprising:
administering to the subject a therapeutically effective amount of a cytotoxic material comprising a compound of claim 1 associated with a cytotoxic agent,
wherein the cytotoxic material is targeted to the tumor in the subject.

59. The method of claim 18, wherein:
the cytotoxic material is injected peritumorally, and
at least a portion of the cytotoxic material is retained at or near the tumor by interactions between the scaffold and a receptor on a surface of a cell in the tumor.
